(19) United States
(12) Patent Application Publication
(10) Pub. No.: US 2009/0081305 A1
(43) Pub. Date: Mar. 26, 2009

(54) Compositions and Methods for Enhancing In-Vivo Uptake of Pharmaceutical Agents

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: Do-Coop Technologies Ltd.

(21) Appl. No.: 12/087,428

(22) PCT Filed: Jan. 4, 2007
(86) PCT No.: PCT/IL2007/000014

§ 371 Date: Jul. 3, 2008

Related U.S. Application Data
(63) Continuation of application No. 11/324,586, filed on Jan. 4, 2006.
Application No. 11/324,586 is a continuation-in-part of application No. PCT/IL05/00198, filed on Feb. 17, 2005.
Application No. 11/324,586 is a continuation-in-part of application No. 10/865,955, filed on Jun. 14, 2004.
Application No. 10/865,955 is a continuation-in-part of application No. PCT/IL02/01004, filed on Dec. 12, 2002.
(60) Provisional application No. 60/755,852, filed on Jan. 4, 2006.
(60) Provisional application No. 60/755,850, filed on Jan. 4, 2006.
(60) Provisional application No. 60/755,851, filed on Jan. 4, 2006.
(60) Provisional application No. 60/545,955, filed on Feb. 20, 2004.

(30) Foreign Application Priority Data
Dec. 12, 2001 (IL) 147049

(51) Int. Cl. A61K 9/16

(52) U.S. Cl. 424/490

(57) ABSTRACT

Pharmaceutical compositions comprising liquid, nanostructures and pharmaceutical agents are provided. Methods of use such compositions are also provided.

### FIELD AND BACKGROUND OF THE INVENTION

The present invention relates to a carrier composition for pharmaceutical agents.

The physiochemical properties of a pharmaceutical agent together with its potency act in concert to determine therapeutic efficacy. For oral and dermal absorption, solubility and lipophilicity are two of the most critical physiochemical properties influencing delivery of a pharmaceutical agent into the systemic circulation [Curatolo W. PSTT. 1998; 1:387-393].

There are also four known mammalian blood barriers including the blood brain barrier (BBB), the blood retinal barrier, the blood testes barrier and the blood mammary gland barrier which function to separate the organ or tissue from activities in the periphery, allowing only selective transport of factors. These provide further obstacles to a pharmaceutical agent from reaching its target site.

Solubility affects the amount of drug available in solution for absorption, and lipophilicity influences the ability of a compound to partition into and across biological membranes including cell membranes and blood barriers. In a large number of cases, there is a strong correlation between these two properties with solubility generally decreasing as lipophilicity increases.

Approximately 40% of newly discovered drugs have little or no water solubility [Connors, R. D. and Elder, E. J., Drug delivery technology: Solubilization Solutions]. This presents a serious challenge to the successful development and commercialization of new drugs in the pharmaceutical industry. No matter how active or potentially active a pharmaceutical agent is against a particular molecular target, if the agent is not available in solution at the site of action, its therapeutic efficacy is negligible. As a result, the development of many pharmaceutical agents is halted before their potential is realized or confirmed, because pharmaceutical companies cannot afford to conduct rigorous preclinical and clinical studies on a molecule that does not have a sufficient pharmacokinetic profile due to poor water solubility.

Improving aqueous solubility is relevant for some already marketed pharmaceutical agents. More than 90% of drugs approved since 1995 have poor solubility, poor permeability, or both. It is estimated that approximately 16% of marketed pharmaceutical agents have less-than-optimal performance specifically because of poor solubility and low bioavailability [Connors, R. D. and Elder, E. J., Drug delivery technology: Solubilization solutions]. The pharmaceutical agent may show performance limitations, such as incomplete or erratic absorption, poor bioavailability, and slow onset of action. Effectiveness can vary from patient to patient, and there can be a strong effect of food on drug absorption. Finally, it may be necessary to increase the dose of a poorly soluble drug to obtain the efficacy required.

Various approaches have been taken to enhance delivery of poorly water-soluble pharmaceutical agents. For example, solid dispersions allow a pharmaceutical agent to be in an amorphous more soluble state due to the presence of diluents such as polyethylene glycol or polyvinylpyrrolidone. However, due to their higher energy state, there is potential for recrystallization.

Microemulsions also aim to enhance delivery of pharmaceutical agents by micellular dispersion of the oil/solvent-dissolved pharmaceutical agent as nanometer size droplets in water. The pharmaceutical agent can be directly absorbed from the droplets. However, there are some concerns about toxicity of high surfactant and co-solvent levels and the possibility of precipitation.

Another approach to pharmaceutical agent delivery is the use of self-emulsifying systems. This involves a mixture of pharmaceutical agent, oil, surfactants and co-solvents that form an emulsion upon administration. Phase inversion may further promote pharmaceutical agent release.

Alternatively, pharmaceutical agents may be reversibly and non-covalently complexed with a “carrier” compound such as cyclodextrin to enhance delivery.

The use of liposomes may be advantageous for enhancing delivery of poorly water soluble pharmaceutical agents into the systemic circulation. This approach involves the encapsulation of a pharmaceutical agent in uni- or multi-layered vesicles of phospholipids. The liposomes can be targeted to specific sites e.g. by using antibody fragments. The liposomes may also act to protect certain pharmaceutical agents from inactivation.

The creation of nanostructured particles of the pharmaceutical agent through particle size reduction and particle formation techniques has also shown to enhance solubility by increasing its surface area.

Nanoparticles have also been used as carriers for pharmaceutical agents. The nanoparticles may incorporate the pharmaceutical agent, e.g. by encapsulation, or alternatively, the pharmaceutical agent may reside between the nanoparticles as taught for example in U.S. Pat. Appl. No. 20030138490.

Poor permeability of pharmaceutical agents across cellular membranes has also been addressed by controlled membrane disruption to allow transient increases in drug transport [Fix, J A. J Pharm Sci. 1996; 85:1282-1285]. However, these technologies often result in indiscriminate, poorly controlled action on membranes that ultimately leads to toleration and safety concerns. An alternative or additional strategy for facilitating translocation of pharmaceutical agents across cellular membranes is the use of membrane transporters [Suzuki H, Sugiyama Y. Eur J Pharm Sci. 2000; 12:3-12]. However, membrane transporters are generally highly specific and much research is required to determine which membrane transporter to target for a particular pharmaceutical agent.

A myriad of devices are also routinely used to aid in pharmaceutical agent delivery to the appropriate site.

For example, to traverse the skin, pharmaceutical agents targeted at internal tissues (i.e., systemic administration) are often administered via transdermal drug delivery systems. Transdermal drug delivery may be targeted to a tissue directly beneath the skin or to capillaries for systemic distribution within the body by blood circulation.

Using a syringe and a needle or other mechanical devices, drugs may be injected into the subcutaneous space thus traversing the epidermis and dermis layers. Although the syringe and needle is an effective delivery device, it is sensitive to contamination, while use thereof is often accompanied by pain and/or bruising. In addition, the use of such a device is accompanied by risk of accidental needle injury to a health care provider. Mechanical injection devices based on compressed gasses have been developed to overcome the above-mentioned limitations of syringe and needle devices. Such devices typically utilize compressed gas (such as, helium or carbon dioxide) to deliver medications at high velocity through a narrow aperture.

Although such devices traverse some of the limitations mentioned above, their efficiency is medication dependent, and their use can lead to pain, bruising and lacerations.

Transdermal drug delivery usually excludes hypodermic injection, long-term needle placement for infusion pumps, and other needles which penetrate the skin's stratum corneum. Thus, transdermal drug delivery is generally regarded as minimally invasive.

Generally, transdermal drug delivery systems employ a medicated device or patch which is affixed to the skin of a patient. The patch allows a pharmaceutical agent contained within it to be absorbed through the skin layers and into the patient's blood stream. Transdermal drug delivery reduces the pain associated with drug injections and intravenous drug administration, as well as the risk of infection associated with these techniques. Transdermal drug delivery also avoids gastrointestinal metabolism of administered drugs, reduces the elimination of drugs by the liver, and provides a sustained release of the administered drug. This type of delivery also enhances patient compliance with a drug regimen because of the relative ease of administration and the sustained release of the drug.

However, many pharmaceutical agents are not suitable for administration via known transdermal drug delivery systems since they are absorbed with difficulty through the skin due to the molecular size of the pharmaceutical agent or to other bioadhesion properties of the agent. In these cases, when transdermal drug delivery is attempted, the drug may be found pooling on the outer surface of the skin and not permeating through the skin into the blood stream.

Generally, conventional transdermal drug delivery methods have been found suitable only for low molecular weight and/or lipophilic drugs such as nitroglycerin for alleviating angina, nicotine for smoking cessation regimens, and estradiol for estrogen replacement in post-menopausal women. Larger pharmaceutical agents such as insulin (a polypeptide for the treatment of diabetes), erythropoietin (used to treat severe anemia) and γ-interferon (used to boost the immune systems cancer fighting ability) are all agents not normally effective when used with conventional transdermal drug delivery methods.

There is thus a widely recognized need for, and it would be highly advantageous to have, a carrier system which is capable of enhancing delivery of pharmaceutical agents devoid of the above limitations.

### SUMMARY OF THE INVENTION

According to the present invention there is provided a pharmaceutical composition comprising at least one pharmaceutical agent as an active ingredient and nanostructures and liquid, wherein the nanostructures comprise a core material of a nanometric size enveloped by ordered fluid molecules of the liquid, the core material and the envelope of ordered fluid molecules being in a steady physical state and whereas the nanostructures and liquid being formulated to enhance in vivo uptake of the at least one pharmaceutical agent.

According to another aspect of the present invention there is provided a method of enhancing in vivo uptake of a pharmaceutical agent into a cell comprising administering the pharmaceutical composition comprising at least one pharmaceutical agent as an active ingredient and nanostructures and liquid, wherein the nanostructures comprise a core material of a nanometric size enveloped by ordered fluid molecules of the liquid, the core material and the envelope of ordered fluid molecules being in a steady physical state and whereas the nanostructures and liquid being formulated to enhance in vivo uptake of the at least one pharmaceutical agent, to an individual, thereby enhancing in vivo uptake of the pharmaceutical agent into the cell.

According to further features in preferred embodiments of the invention described below, the pharmaceutical agent is a therapeutic agent, cosmetic agent or a diagnostic agent.

According to still further features in the described preferred embodiments the therapeutic agent is selected from the group consisting of an antibiotic agent, an analeptic agent, an anti-convulsant agent, an anti-neoplastic agent, an anti-inflammatory agent, an antiparasitic agent, an antifungal agent, an antimycobacterial agent, an antiviral agent, an antihistamine agent, an anticoagulant agent, a radiotherapeutic agent, a chemotherapeutic agent, a cytotoxic agent, a neurotrophic agent, a psychotherapeutic agent, an anxiolytic sedative agent, a stimulant agent, a sedative agent, an analgesic agent, an anesthetic agent, a vasodilating agent, a birth control agent, a neurotransmitter agent, a neurotransmitter analog agent, a scavenging agent, a fertility-enhancing agent and an anti-oxidant agent.

According to still further features in the described preferred embodiments, the neurotransmitter agent is selected from the group consisting of acetylcholine, dopamine, norepinephrine, serotonin, histamine, epinephrine, Gamma-aminobutyric acid (GABA), glycine, glutamate, adenosine, inosine and aspartate.

According to still further features in the described preferred embodiments, the pharmaceutical agent is selected from the group consisting of a protein agent, a nucleic acid agent, a small molecule agent, a cellular agent and a combination thereof.

According to still further features in the described preferred embodiments, the protein agent is a peptide.

According to still further features in the described preferred embodiments, the protein agent is selected from the group consisting of an enzyme, a growth factor, a hormone and an antibody.

According to still further features in the described preferred embodiments, the peptide is a neuropeptide.

According to still further features in the described preferred embodiments, the neuropeptide is selected from the group consisting of Oxytocin, Vasopressin, Corticotropin releasing hormone (CRH), Growth hormone releasing hormone (GHRH), Luteinizing hormone releasing hormone (LHRH), Somatostatin growth hormone release inhibiting hormone, Thyrotropin releasing hormone (TRH), Neurokinin a (substance K), Neurokinin β, Neuropeptide K, Substance P, β-endorphin, Dynorphin, Met- and leu-enkephalin, Neuropeptide tyrosine (NPY), Pancreatic polypeptide, Peptide tyrosine-tyrosine (PYY), Glucogen-like peptide-1 (GLP-1), Peptide histidine isoleucine (PHI), Pituitary adenylate cyclase activating peptide (PACAP), Vasoactive intestinal polypeptide (VIP), Brain natriuretic peptide, Calcitonin gene-related peptide (CGRP) (α- and β-form), Cholecystokinin (CCK), Galanin, Islet amyloid polypeptide (IAPP), Melanin concentrating hormone (MCH), ACTH, α-MSH, Neuropeptide FF, Neurotensin, Parathyroid hormone related protein, Agouti gene-related protein (AGRP), Cocaine and amphetamine regulated transcript (CART)/peptide, Endomorphin-1 and -2,5-HT-moduline, Hypocretins/orexins Nociceptin/orphanin FQ, Nocistatin, Prolactin releasing peptide, Secretoneurin and Urocortin.

According to still further features in the described preferred embodiments, the cellular agent is a virus.

According to still further features in the described preferred embodiments, the virus is a bacteriophage.

According to still further features in the described preferred embodiments, the small molecule agent has a molecular mass of less than 1000 Da.

According to still further features in the described preferred embodiments, the diagnostic agent is a contrast agent.

According to still further features in the described preferred embodiments, the contrast agent is selected from the group consisting of an X-ray imaging contrast agent, a magnetic resonance imaging contrast agent and an ultrasound imaging contrast agent.

According to still further features in the described preferred embodiments, the diagnostic agent is a radioimaging agent or a fluorescence imaging agent.

According to still further features in the described preferred embodiments, at least a portion of the fluid molecules are in a gaseous state.

According to still further features in the described preferred embodiments, a concentration of the nanostructures is less than 10²⁰ per liter.

According to still further features in the described preferred embodiments, a concentration of the nanostructures is less than 10¹⁵ per liter.

According to still further features in the described preferred embodiments, the nanostructures are capable of forming clusters.

According to still further features in the described preferred embodiments, the nanostructures are capable of maintaining long range interaction thereamongst.

According to still further features in the described preferred embodiments, the nanostructures and liquid is characterized by an enhanced ultrasonic velocity relative to water.

According to still further features in the described preferred embodiments, the core material is selected from the group consisting of a ferroelectric material, a ferromagnetic material and a piezoelectric material.

According to still further features in the described preferred embodiments, the core material is a crystalline core material.

According to still further features in the described preferred embodiments, the liquid is water.

According to still further features in the described preferred embodiments, the nanostructures is characterized by a specific gravity lower than or equal to a specific gravity of the liquid.

According to still further features in the described preferred embodiments, the nanostructures and liquid comprise a buffering capacity greater than a buffering capacity of water.

According to still further features in the described preferred embodiments, the nanostructures are formulated from hydroxyapatite.

According to still further features in the described preferred embodiments, the therapeutic agent is selected to treat a skin condition.

According to still further features in the described preferred embodiments, the skin condition is selected from the group consisting of acne, psoriasis, vitiligo, a keloid, a burn, a scar, a wrinkle, xerosis, ichthyosis, keratosis, keratoderma, dermatitis, pruritis, eczema, skin cancer, a hemorrhoid and a callus.

According to still further features in the described preferred embodiments, the pharmaceutical composition is formulated in a topical composition.

According to still further features in the described preferred embodiments, the pharmaceutical agent is selected to treat or diagnose a brain condition.

According to still further features in the described preferred embodiments, the brain condition is selected from the group consisting of brain tumor, neuropathy, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

According to still further features in the described preferred embodiments, the cell is a mammalian cell, a bacterial cell or a viral cell.

The present invention successfully addresses the shortcomings of the presently known configurations by providing a carrier composition which enhances the in vivo uptake of pharmaceutical agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph representing the number of colony forming units (CFU) of electrically competent E. coli bacteria resuspended in standard solution (90% water, 10% glycerol) or increasing concentrations of the carrier composition and glycerol. The numbers represent mean values+STD obtained from at least 3 independent experiments.

FIG. 2 is a bar graph representing the transformation efficiency of three different chemically competent bacteria strains transformed with pUC plasmid DNA and diluted 1:10 in either water or the carrier composition. The results are presented as the ratio between the CFU obtained in carrier composition-plates and those of control.

FIGS. 3A-B are photographs of fluorescent microscopy images 48 hours following transfection of a green fluorescent protein (GFP) construct into primary human cells. FIG. 3A depicts transfection using lipofectamine. FIG. 3B depicts transfection using lipofectamine together with the carrier composition.

FIGS. 4A-B are photographs of agar plates containing a bacterial lawn of S. aureus following spotting of Phage strain #6. FIG. 4A is a photograph of carrier composition-based agar plate. FIG. 4B is a photograph of a control plate. The numbers (1-8) represent 100-fold serial dilutions of phage RTD. The arrows point to the presence (FIG. 4A) or absence (FIG. 4B) of plaque in dilution #3.

FIGS. 5A-D are photographs of agar plates containing a bacterial lawn of S. aureus following spotting of Phage strain #83A (FIGS. 5A-B) and Phage strain #6 (FIGS. 5C-D) and incubation for three hours at 37° C. FIGS. 5A and 5C are photographs of carrier composition-based agar plates. FIGS. 5B and 5D are photographs of control plates.

FIG. 6 is a bar graph illustrating phage strain #6 and #83A infection of S. aureus in either control or carrier composition LB broth. Optical density (OD) of bacteria-phage broth was measured when lysis was apparent (time 0) and at different time intervals as indicated.

FIG. 7 is a graph illustrating the number of plaque forming units (pfu) obtained following addition of dilutions of phage λ GEM 11 to a competent bacterial host. Dilutions were performed with either control or carrier composition-based SM buffer in series of 1/10 dilutions.

FIGS. 8A-B are photographs of agar plates comprising Bacillus subtilis bacterial colonies pre-grown in the presence (FIG. 8B) and absence (FIG. 8A) of the carrier composition.

FIGS. 9A-C are photographs of agar plates comprising 10̂5 bacterial colonies pre-grown in the presence (FIG. 9C) and absence (FIG. 9B) of the carrier composition and in the presence of SP water (reverse osmosis-water mixed with the same source powder as in the carrier composition —FIG. 9A).

FIGS. 10A-C are photographs of agar plates comprising T strain bacterial colonies pre-grown in the presence (FIG. 10C) and absence (FIGS. 10A-B) of the carrier composition both in the presence (FIGS. 10B-C) and absence (FIG. 10A) of streptomycin.

FIG. 11 is a plot graph demonstrating the turbidity of Vibrio Harveyi bacteria grown in distilled water or carrier composition over time.

FIG. 12 is a plot graph demonstrating the luminescence of Vibrio Harveyi bacteria grown in distilled water or carrier composition over time.

FIGS. 13A-C are photographs of an identical woman following a three day treatment of a dermal cream diluted in the carrier composition and computer read-outs indicating the number of spots [red spots indicate a first-stage infection, and yellow spots indicate a second, more advanced stage of infection] she has on a marked area of her skin. FIG. 13A is a photograph and read-out following one day of treatment. FIG. 13B is a photograph and read-out following two days of treatment. FIG. 13C is a photograph and read-out following three days of treatment.

FIG. 14 shows results of isothermal measurement of absolute ultrasonic velocity in the liquid composition of the present invention as a function of observation time.

FIG. 15 is a photograph of a plastic apparatus comprising four upper channels and one lower channel connected via capillary channels.

FIGS. 16A-B are photographs of plastic apparatus following addition of a dye and diluting agent to the upper channels. FIG. 16A shows that fifteen minutes following placement there is no movement from the upper channels to the lower channel via the capillaries when the diluting agent is water. FIG. 16B shows that fifteen minutes following placement, there is movement from the upper channels to the lower channel via the capillaries when the diluting agent is the liquid composition of the present invention.

FIG. 17 is a graph illustrating sodium hydroxide titration of various water compositions as measured by absorbence at 557 nm.

FIGS. 18A-C are graphs of an experiment performed in triplicate illustrating Sodium hydroxide titration of water comprising nanostructures and RO water as measured by pH.

FIGS. 19A-C are graphs illustrating Sodium hydroxide titration of water comprising nanostructures and RO water as measured by pH, each graph summarizing 3 triplicate experiments.

FIGS. 20A-C are graphs of an experiment performed in triplicate illustrating Hydrochloric acid titration of water comprising nanostructures and RO water as measured by pH.

FIG. 21 is a graph illustrating Hydrochloric acid titration of water comprising nanostructures and RO water as measured by pH, the graph summarizing 3 triplicate experiments.

FIGS. 22A-C are graphs illustrating Hydrochloric acid (FIG. 22A) and Sodium hydroxide (FIGS. 22B-C) titration of water comprising nanostructures and RO water as measured by absorbence at 557 nm.

FIGS. 23A-B are photographs of cuvettes following Hydrochloric acid titration of RO (FIG. 23A) and water comprising nanostructures (FIG. 23B). Each cuvette illustrated addition of 1 μl of Hydrochloric acid.

FIGS. 24A-C are graphs illustrating Hydrochloric acid titration of RF water (FIG. 24A), RF2 water (FIG. 24B) and RO water (FIG. 24C). The arrows point to the second radiation.

FIG. 25 is a graph illustrating Hydrochloric acid titration of RF2 water as compared to RO water. The experiment was repeated three times. An average value for all three experiments was plotted for RO water.

FIGS. 26A-J are photographs of solutions comprising red powder and Neowater™ following three attempts at dispersion of the powder at various time intervals. FIGS. 26A-E illustrate right test tube C (50% EtOH+Neowater™) and left test tube B (dehydrated Neowater™) from Example 14, part A. FIGS. 26G-J illustrate solutions following overnight crushing of the red powder and titration of 100 μl Neowater™

FIGS. 27A-C are readouts of absorbance of 2 μl from 3 different solutions as measured in a nanodrop. FIG. 27A represents a solution of the red powder following overnight crushing+100 μl Neowater. FIG. 27B represents a solution of the red powder following addition of 100% dehydrated Neowater™ and FIG. 27C—represents a solution of the red powder following addition of EtOH+Neowater™ (50%-50%).

FIG. 28 is a graph of spectrophotometer measurements of vial #1 (CD-Dau+Neowater™), vial #4 (CD-Dau+10% PEG in Neowater™) and vial #5 (CD-Dau+50% Acetone+50% Neowater™).

FIG. 29 is a graph of spectrophotometer measurements of the dissolved material in Neowater™ (blue line) and the dissolved material with a trace of the solvent acetone (pink line).

FIG. 30 is a graph of spectrophotometer measurements of the dissolved material in Neowater™ (blue line) and acetone (pink line). The pale blue and the yellow lines represent different percent of acetone evaporation and the purple line is the solution without acetone.

FIG. 31 is a graph of spectrophotometer measurements of CD-Dau at 200-800 nm. The blue line represents the dissolved material in RO while the pink line represents the dissolved material in Neowater™.

FIG. 32 is a graph of spectrophotometer measurements of t-boc at 200-800 nm. The blue line represents the dissolved material in RO while the pink line represents the dissolved material in Neowater™.

FIGS. 33A-D are graphs of spectrophotometer measurements at 200-800 nm. FIG. 33A is a graph of AG-14B in the presence and absence of ethanol immediately following ethanol evaporation. FIG. 33B is a graph of AG-14B in the presence and absence of ethanol 24 hours following ethanol evaporation. FIG. 33C is a graph of AG-14A in the presence and absence of ethanol immediately following ethanol evaporation. FIG. 33D is a graph of AG-14A in the presence and absence of ethanol 24 hours following ethanol evaporation.

FIG. 34 is a photograph of suspensions of AG-14A and AG14B 24 hours following evaporation of the ethanol.

FIGS. 35A-G are graphs of spectrophotometer measurements of the peptides dissolved in Neowater™. FIG. 35A is a graph of Peptide X dissolved in Neowater™. FIG. 35B is a graph of X-5FU dissolved in Neowater™. FIG. 35C is a graph of NLS-E dissolved in Neowater™. FIG. 35D is a graph of Palm-PFPSYK (CMFU) dissolved in Neowater™. FIG. 35E is a graph of PFPSYKLRPG-NH₂ dissolved in Neowater™. FIG. 35F is a graph of NLS-p2-LHRH dissolved in Neowater™, and FIG. 35G is a graph of F-LH-RH-palm kGFPSK dissolved in Neowater™.

FIGS. 36A-G are bar graphs illustrating the cytotoxic effects of the peptides dissolved in Neowater™ as measured by a crystal violet assay. FIG. 36A is a graph of the cytotoxic effect of Peptide X dissolved in Neowater™. FIG. 36B is a graph of the cytotoxic effect of X-5FU dissolved in Neowater™. FIG. 36C is a graph of the cytotoxic effect of NLS-E dissolved in Neowater™. FIG. 36D is a graph of the cytotoxic effect of Palm-PFPSYK (CMFU) dissolved in Neowater™. FIG. 36E is a graph of the cytotoxic effect of PFPSYKLRPG-NH₂ dissolved in Neowater™. FIG. 36F is a graph of the cytotoxic effect of NLS-p2-LHRH dissolved in Neowater™, and FIG. 36G is a graph of the cytotoxic effect of F-LH-RH-palm kGFPSK dissolved in Neowater™.

FIG. 37 is a graph of retinol absorbance in ethanol and Neowater™.

FIG. 38 is a graph of retinol absorbance in ethanol and Neowater™ following filtration.

FIGS. 39A-B are photographs of test tubes, the left containing Neowater™ and substance “X” and the right containing DMSO and substance “X”. FIG. 39A illustrates test tubes that were left to stand for 24 hours and FIG. 39B illustrates test tubes that were left to stand for 48 hours.

FIGS. 40A-C are photographs of test tubes comprising substance “X” with solvents 1 and 2 (FIG. 40A), substance “X” with solvents 3 and 4 (FIG. 40B) and substance “X” with solvents 5 and 6 (FIG. 40C) immediately following the heating and shaking procedure.

FIGS. 41A-C are photographs of test tubes comprising substance “X” with solvents 1 and 2 (FIG. 41A), substance “X” with solvents 3 and 4 (FIG. 41B) and substance “X” with solvents 5 and 6 (FIG. 41C) 60 minutes following the heating and shaking procedure.

FIGS. 42A-C are photographs of test tubes comprising substance “X” with solvents 1 and 2 (FIG. 42A), substance “X” with solvents 3 and 4 (FIG. 42B) and substance “X” with solvents 5 and 6 (FIG. 42C) 120 minutes following the heating and shaking procedure.

FIGS. 43A-C are photographs of test tubes comprising substance “X” with solvents 1 and 2 (FIG. 43A), substance “X” with solvents 3 and 4 (FIG. 43B) and substance “X” with solvents 5 and 6 (FIG. 43C) 24 hours following the heating and shaking procedure.

FIGS. 44A-D are photographs of glass bottles comprising substance “X” in a solvent comprising Neowater™ and a reduced concentration of DMSO, immediately following shaking (FIG. 44A), 30 minutes following shaking (FIG. 44B), 60 minutes following shaking (FIG. 44C) and 120 minutes following shaking (FIG. 44D).

FIG. 45 is a graph illustrating the absorption characteristics of material “X” in RO/Neowater™ 6 hours following vortex, as measured by a spectrophotometer.

FIGS. 46A-B are graphs illustrating the absorption characteristics of SPL2101 in ethanol (FIG. 46A) and SPL5217 in acetone (FIG. 46B), as measured by a spectrophotometer.

FIGS. 47A-B are graphs illustrating the absorption characteristics of SPL2101 in Neowater™ (FIG. 47A) and SPL5217 in Neowater™ (FIG. 47B), as measured by a spectrophotometer.

FIGS. 48A-B are graphs illustrating the absorption characteristics of taxol in Neowater™ (FIG. 48A) and DMSO (FIG. 48B), as measured by a spectrophotometer.

FIG. 49 is a bar graph illustrating the cytotoxic effect of taxol in different solvents on 293T cells. Control RO=medium made up with RO water; Control Neo=medium made up with Neowater™; Control DMSO RO=medium made up with RO water+10 μl DMSO; Control Neo RO=medium made up with RO water+10 μl Neowater™; Taxol DMSO RO=medium made up with RO water+taxol dissolved in DMSO; Taxol DMSO Neo=medium made up with Neowater™+taxol dissolved in DMSO; Taxol NW RO=medium made up with RO water+taxol dissolved in Neowater™; Taxol NW Neo=medium made up with Neowater™+taxol dissolved in Neowater™.

FIGS. 50A-B are photographs of a DNA gel stained with ethidium bromide illustrating the PCR products obtained in the presence and absence of the liquid composition comprising nanostructures following heating according to the protocol described in Example 22 using two different Taq polymerases.

FIG. 51 is a photograph of a DNA gel stained with ethidium bromide illustrating the PCR products obtained in the presence and absence of the liquid composition comprising nanostructures following heating according to the protocol described in Example 23 using two different Taq polymerases.

### DESCRIPTION OF THE PREFERRED EMBODIMENTS

The present invention is of carrier compositions which can enhance the in-vivo uptake of pharmaceutical agents.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The development of many pharmaceutical agents with low bioavailability such as peptides, proteins and nucleic acids has created a need to develop new and effective approaches of delivering such macromolecules to their appropriate cellular targets. Therapeutics based on either the use of specific polypeptide growth factors or specific genes to replace or supplement absent or defective genes are examples of therapeutics that require such new delivery systems. Therapeutic agents involving oligonucleotides such that they interact with DNA to modulate the expression of a gene may also require a delivery system that is capable of enhancing in vivo uptake across cellular membranes. Clinical application of such therapies depends not only on the reliability and efficiency of new delivery systems but also on their safety and on the ease with which the technologies underlying these systems can be adapted for large-scale pharmaceutical production, storage, and distribution of the therapeutic formulations.

Nanoparticle technology has found application in a variety of disciplines, but has only minimal application in pharmacology and drug delivery. Nanoparticles have been proposed as carriers of anticancer and other drugs [Couvreur et al., (1982) J. Pharm. Sci., 71: 790-92]. Other attempts have pursued the use of nanoparticles for treatment of specific disorders [Labhasetwar et al., (1997) Adv. Drug. Del. Rev., 24: 63-85]. Typically, the nanoparticles are loaded with the pharmaceutical agent.

Although nanoparticles have shown promise as useful tools for drug delivery systems, many problems remain. Some unsolved problems relate to the loading of particles with therapeutics. Additionally, the bioavailability of loaded nanoparticles is reduced since nanoparticles are taken up by cell of the reticuloendothelial system (RES). Therefore, it would be highly advantageous to have a nanoparticle delivery system which is devoid of the above limitations.

While reducing the present invention to practice, the present inventor has uncovered that a carrier composition comprising nanostructures (such as those described in U.S. Pat. Appl. No. 60/545,955 and Ser. No. 10/865,955, and International Patent Application, Publication No. WO2005/079153) can be used to efficiently enhance in vivo cellular uptake of a pharmaceutical agent.

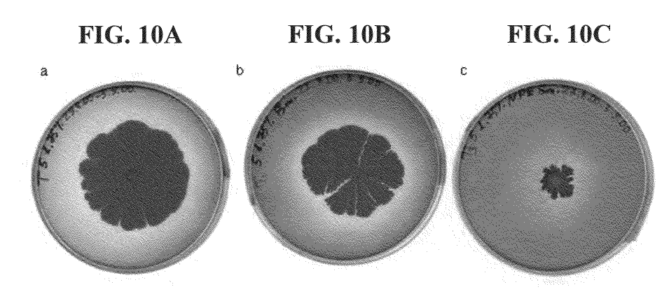

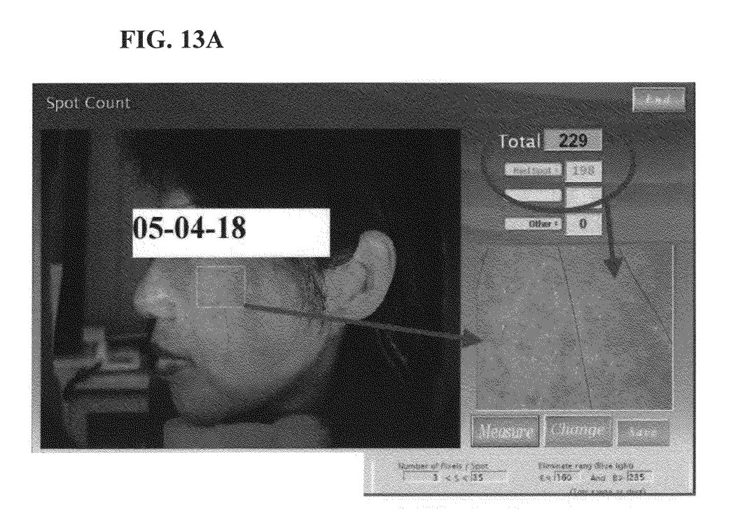

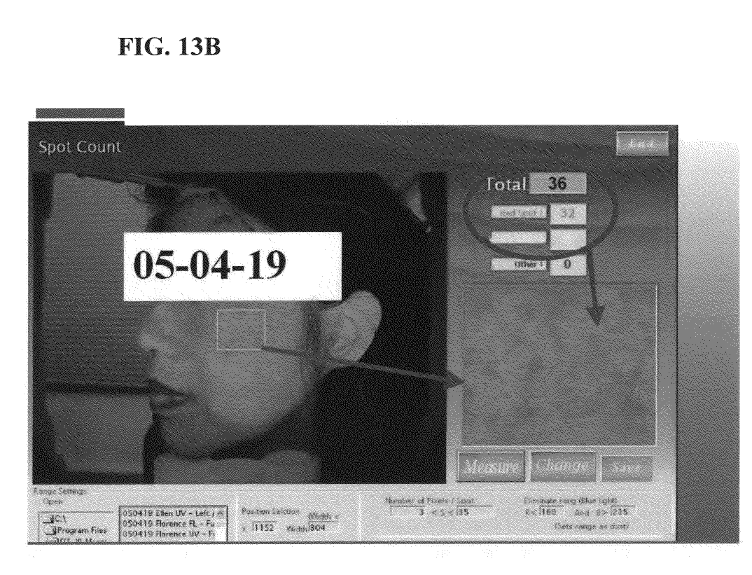

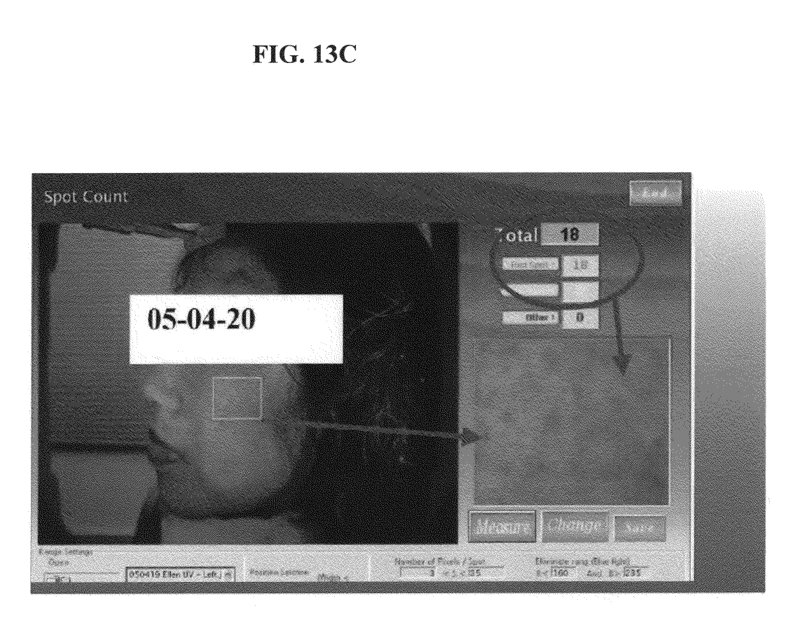

As illustrated hereinbelow and in the Examples section which follows the present inventor has demonstrated that the above-mentioned nanostructures and liquid can enhance in vivo penetration of a therapeutic agent through cell membranes. For example, a carrier composition comprising nanostructures and liquid was shown to enhance penetration of a therapeutic agent through the skin (FIGS. 13A-C). Additionally, the carrier composition was shown to enhance uptake of an antibiotic agent into bacteria cells, thereby increasing its bioavailability (FIGS. 10A-C).

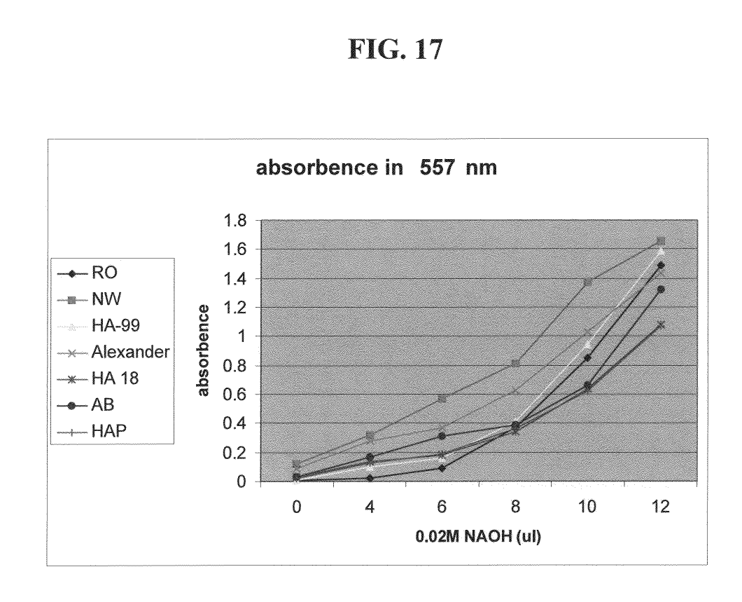

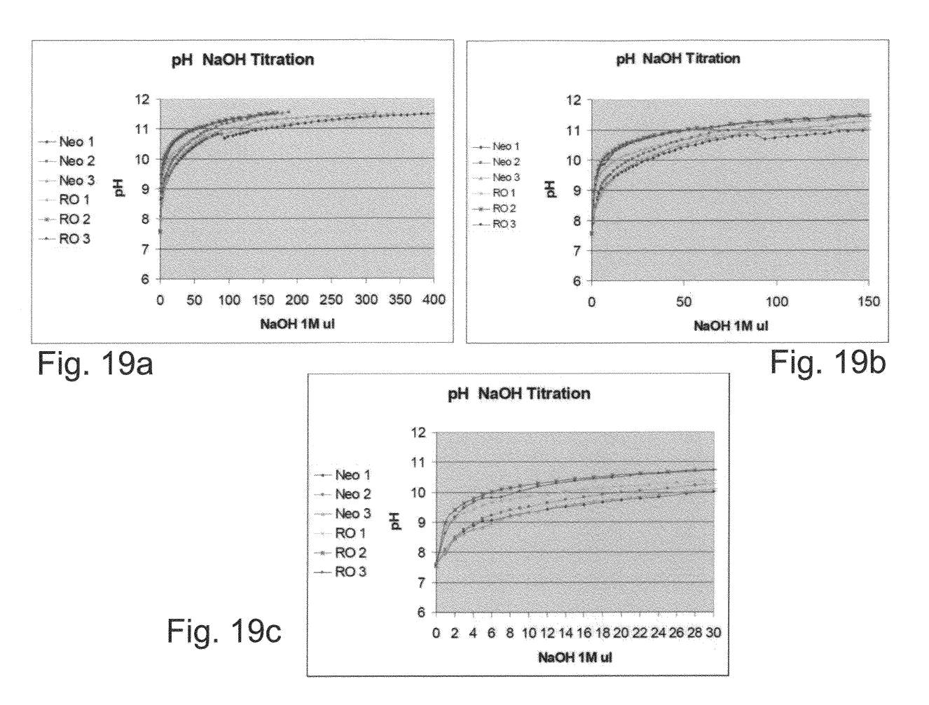

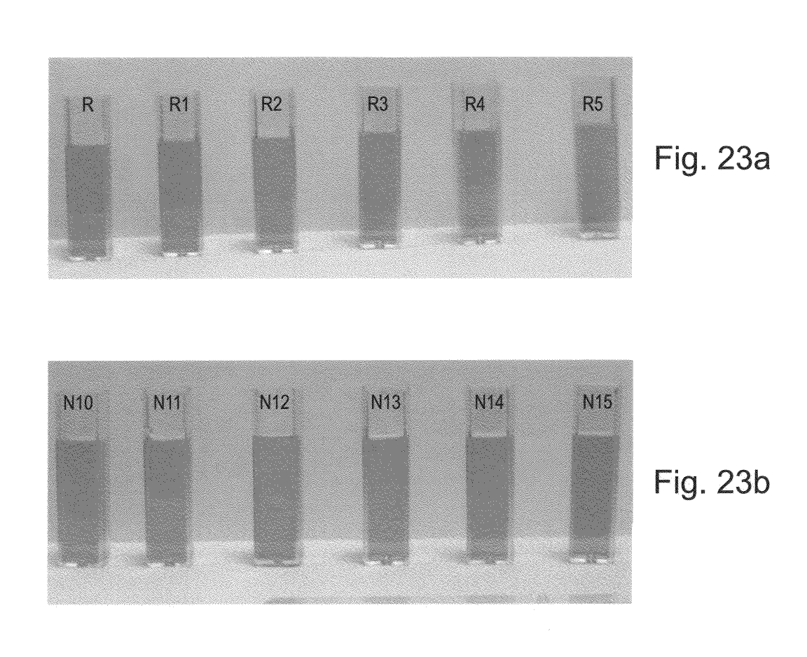

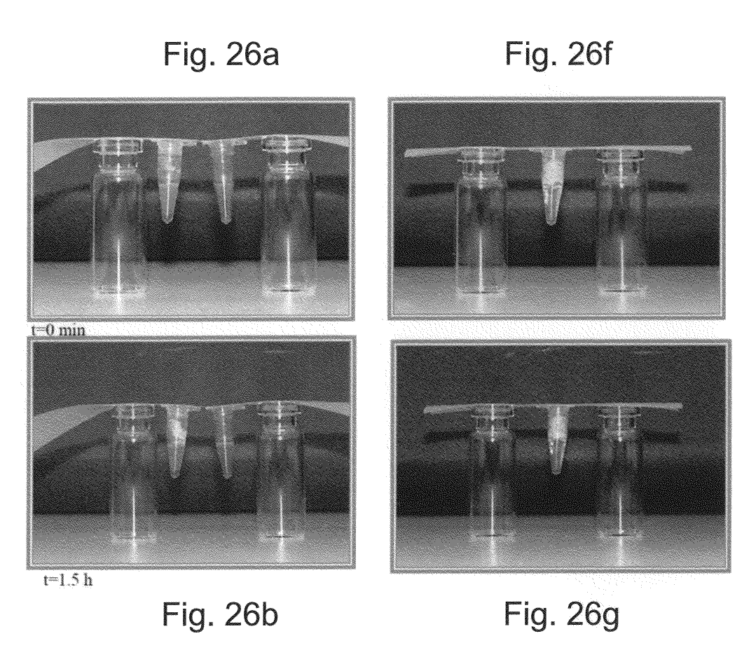

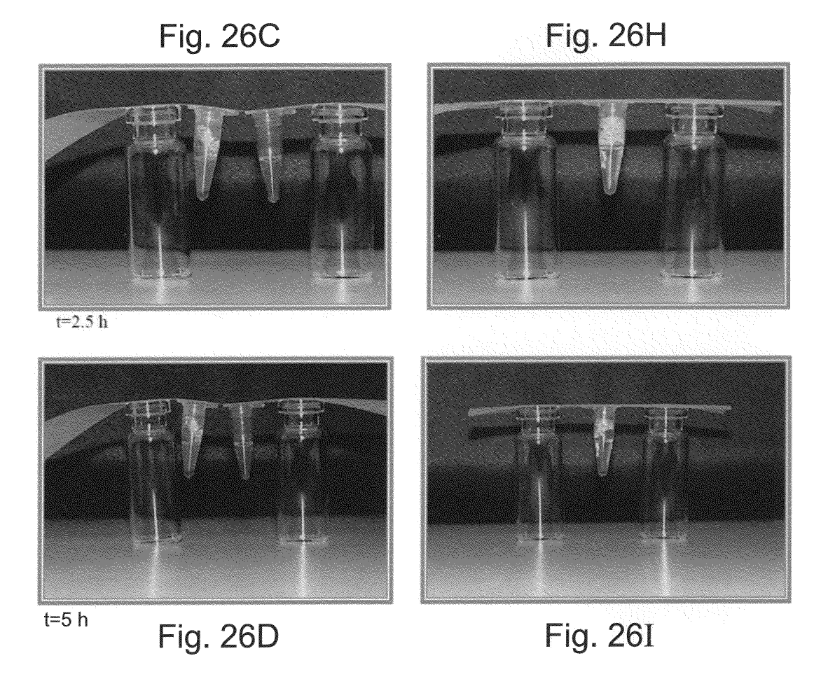

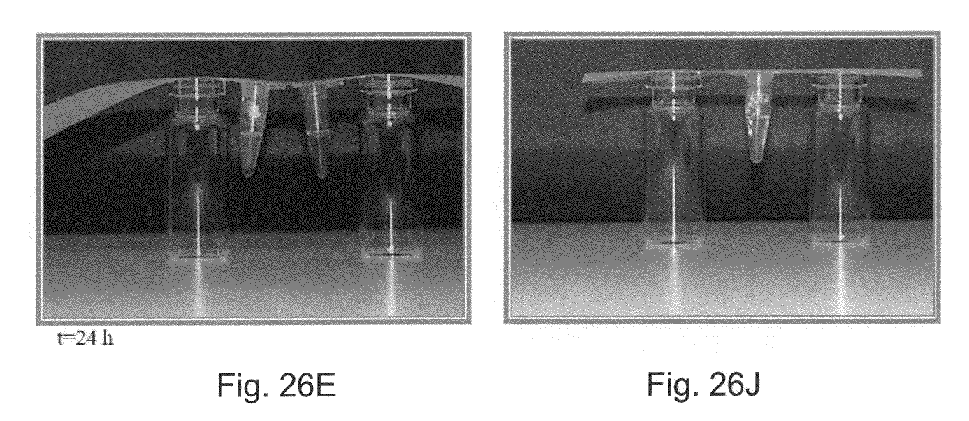

Furthermore, the present inventors have demonstrated that the carrier composition of the present invention comprises an enhanced ability to both dissolve and disperse agents which are not readily dissolvable in water (FIGS. 26-49). In addition, the present inventors have shown that the carrier composition of the present invention comprises a buffering capacity (FIGS. 17-25) and is capable of stabilizing a peptide agent. All of these attributes contribute to the ability of the composition of the present invention to enhance in-vivo uptake.

Thus, according to one aspect of the present invention there is provided a pharmaceutical composition comprising at least one pharmaceutical agent as an active ingredient and nanostructures and liquid. The nanostructures comprise a core material of a nanometric size enveloped by ordered fluid molecules of the liquid and the core material and the envelope of the ordered fluid molecules are in a steady physical state. The nanostructures and liquid are formulated to enhance in vivo uptake of the at least one pharmaceutical agent (i.e., carrier).

As used herein the phrase “pharmaceutical agent as an active ingredient” refers to a therapeutic, cosmetic or diagnostic agent which is accountable for the biological effect of the pharmaceutical composition.

As used herein a “pharmaceutical composition” refers to a preparation of one or more of the active ingredients with the carrier composition, both described herein.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, or between several hundreds of picometers to several hundreds of nanometers. Thus, the nanostructure of the present embodiments can comprise a nanoparticle, an arrangement of nanoparticles, or any arrangement of one or more nanoparticles and one or more molecules.

The liquid of the above described composition is preferably an aquatic liquid e.g., water.

According to this aspect of the present invention the nanostructures of the pharmaceutical composition of the present invention comprise a core material of a nanometer size enveloped by ordered fluid molecules, which are in a steady physical state with each other.

Examples of core materials include, without being limited to, a ferroelectric material, a ferromagnetic material and a piezoelectric material. A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. Preferably, the nanostructures retains the ferroelectric or ferromagnetic properties of the core material, thereby incorporating a particular feature in which macro scale physical properties are brought into a nanoscale environment.

The core material may also have a crystalline structure.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

As used herein, the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the carrier composition. The fluid molecules of the envelope may comprise an additional fluid which is not identical to the liquid molecules of the carrier composition and as such the envelope may comprise a heterogeneous fluid composition.

Due to the formation of the envelope of ordered fluid molecules, the nanostructures of the present embodiment preferably have a specific gravity which is lower than or equal to a specific gravity of the liquid.

The fluid molecules may be either in a liquid state or in a gaseous state or a mixture of the two.

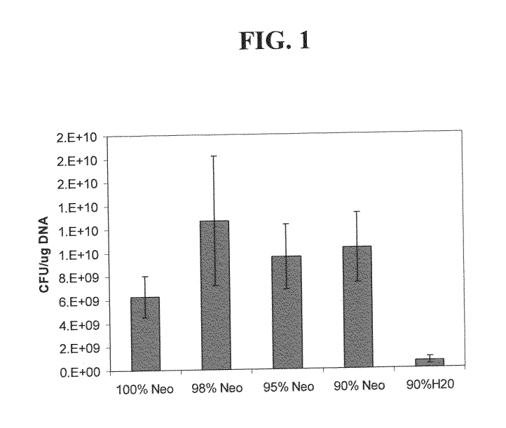

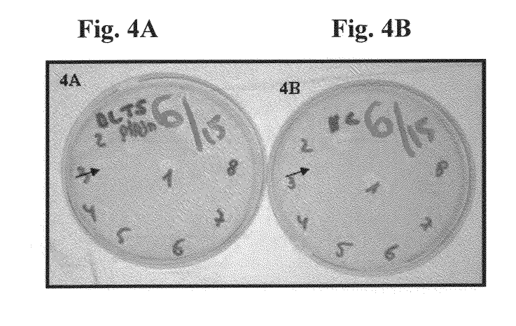

According to this aspect of the present invention the nanostructures and liquid are formulated to enhance in vivo uptake of the pharmaceutical agent. Without being bound to theory, it is believed that the long-range interactions between the nanostructures lends to the unique characteristics of the pharmaceutical compositions of the present invention. One such characteristic is that the carrier composition of the present invention is hydrophobic as demonstrated in Example 9 and is thus able to enhance penetration of an active agent through cellular membranes membrane. For example, as demonstrated in Examples 1, 2 and 3, the carrier composition of the present invention enhances nucleotide uptake into cells (FIGS. 1, 2 and 3A-B). Additionally, the carrier composition of the present invention enhances phage uptake (FIGS. 4A-B, 5A-D, 6 and 7) and antibiotic uptake (FIGS. 10A-C) into bacterial cells.

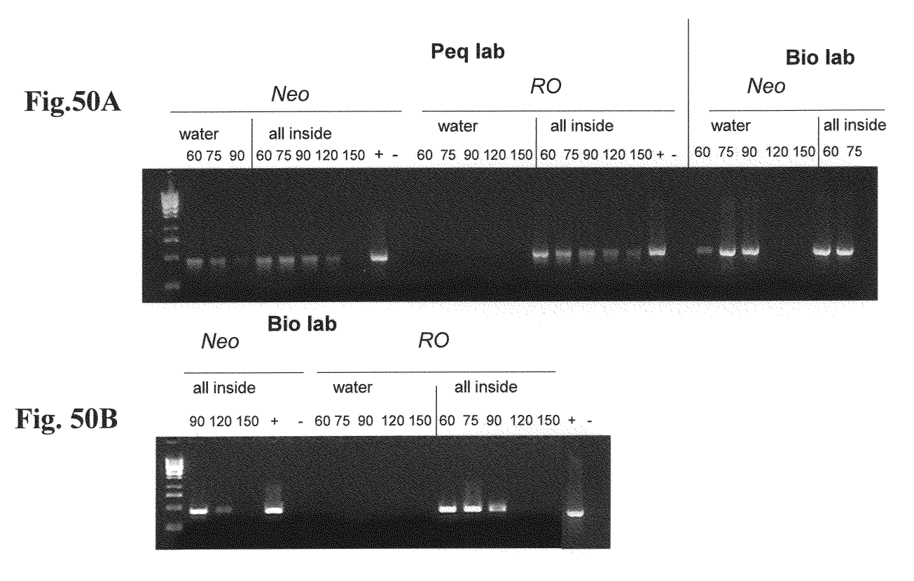

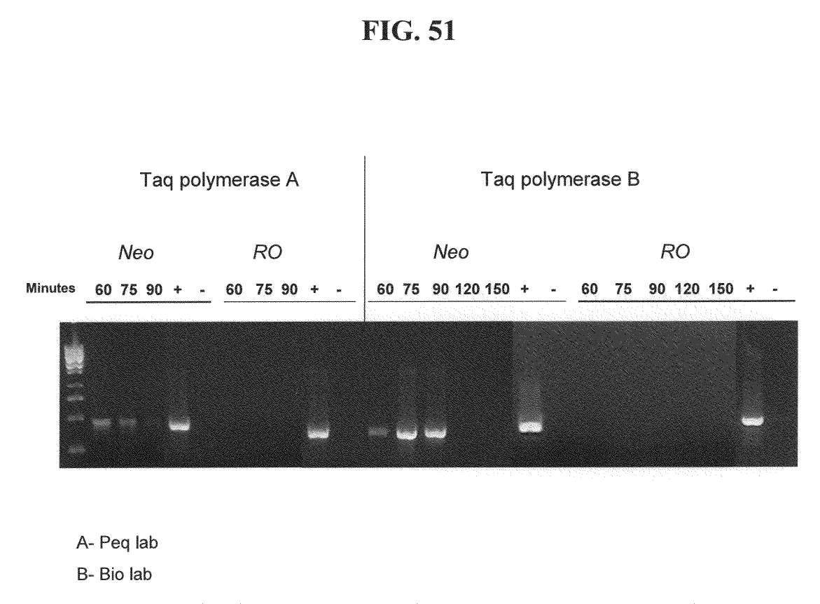

The carrier composition may also enhance in vivo uptake of a pharmaceutical agent by increasing its solubility and/or dispersion (FIGS. 26-49). Additionally, or alternatively, the carrier composition may enhance in vivo uptake of a pharmaceutical agent by providing thereto a stabilizing environment. For example, it has been shown that the carrier composition is capable of stabilizing proteins (FIGS. 50A-B and FIG. 51).

Furthermore, the present inventors have shown that the composition of the present invention comprises a buffering capacity greater than a buffering capacity of water (FIGS. 17-25).

As used herein, the phrase “buffering capacity” refers to the composition's ability to maintain a stable pH stable as acids or bases are added.

Thus, the nanostructures and liquid may be formulated to enhance penetration is through any biological barrier such as a cell membrane, an organelle membrane, a blood barrier or a tissue. For example the nanostructures and liquid may be formulated to penetrate the skin (Example 7—FIGS. 13A-C).

A preferred concentration of nanostructures is below 10²⁰ nanostructures per liter and more preferably below 10¹⁵ nanostructures per liter. The concentration of nanostructures is preferably selected according to the intended use as described herein below.

Preferably the nanostructures in the liquid are capable of clustering due to attractive electrostatic forces between them. Preferably, even when the distance between the nanostructures prevents cluster formation (about 0.5-10 μm), the nanostructures are capable of maintaining long range interactions.

The long range interaction of the nanostructures has been demonstrated by the present Inventor (see Example 7 in the Examples section that follows). The carrier composition of the present embodiment was subjected to temperature changes and the effect of temperature change on ultrasonic velocity was investigated. As will be appreciated by one of ordinary skill in the art, ultrasonic velocity is related to the interaction between the nanostructures in the composition. As demonstrated in the Examples section that follows, the carrier composition of the present invention is characterized by an enhanced ultrasonic velocity relative to water.

Production of the nanostructures according to this aspect of the present invention may be carried out using a “top-down” process. The process comprises the following method steps, in which a solid powder (e.g., a mineral, a ceramic powder, a glass powder, a metal powder, or a synthetic polymer) is heated, to a sufficiently high temperature, preferably more than about 700° C. Examples of solid powders which are contemplated include, but are not limited to, BaTiO₃, WO₃ and Ba₂F₉O₁₂.

Examples of solid powders which are contemplated include, but are not limited to, BaTiO₃, WO₃ and Ba₂F₉O₁₂. Surprisingly, the present inventors have also shown that hydroxyapatite (HA) may be heated to produce the liquid composition of the present invention.

Hydroxyapatite is specifically preferred as it is characterized by intoxocicty and is generally FDA approved for human therapy.

It will be appreciated that many hydroxyapatite powders are available from a variety of manufacturers such as from Sigma Aldrich and Clarion Pharmaceuticals (e.g. Catalogue No. 1306-06-5).

As shown in Table 2, liquid compositions based on HA, all comprised enhanced buffering capacities as compared to water.

The heated powder is then immersed in a cold liquid, below its density anomaly temperature, e.g., 3° C. or 2° C. Simultaneously, the cold liquid and the powder are irradiated by electromagnetic RF radiation, preferably above 500 MHz, which may be either continuous wave RF radiation or modulated RF radiation.

As mentioned, the pharmaceutical agent may be a therapeutic agent, a cosmetic agent or a diagnostic agent.

Examples of structural classes of therapeutic agents include, but are not limited to, inorganic or organic compounds; small molecules (i.e., less than 1000 Daltons) or large molecules (i.e., above 1000 Daltons); biomolecules (e.g. proteinaceous molecules, including, but not limited to, protein (e.g. enzymes or hormones) peptide, polypeptide, post-translationally modified protein, antibodies etc.) or nucleic acid molecules (e.g. double-stranded DNA, single-stranded DNA, double-stranded RNA, single-stranded RNA, or triple helix nucleic acid molecules) or chemicals. Therapeutic agents may be cellular agents derived from any known organism (including, but not limited to, animals, plants, bacteria, fungi, protista or viruses) or from a library of synthetic molecules. An example of a viral therapeutic cellular agent is a bacteriophage. As demonstrated in Example 4 of the Examples section which follows and in FIGS. 4A-B, 5A-D, 6 and 7, the carrier composition of the present invention enabled increased bacteriophage uptake into bacteria.

Examples of therapeutic agents which may be particularly useful in treating a brain condition include, but are not limited to antibiotic agents, anti-neoplastic agents, anti-inflammatory agents, antiparasitic agents, antifungal agents, antimycobacterial agents, antiviral agents, anticoagulant agents, radiotherapeutic agents, chemotherapeutic agents, cytotoxic agents, vasodilating agents, anti-oxidants, analeptic agents, anti-convulsant agents, antihistamine agents, neurotrophic agents, psychotherapeutic agents, anxiolytic sedative agents, stimulant agents, sedative agents, analgesic agents, anesthetic agents, birth control agents, neurotransmitter agents, neurotransmitter analog agents, scavenging agents and fertility-enhancing agents.

Examples of neurotransmitter agents which can be used in accordance with the present invention include but are not limited to acetylcholine, dopamine, norepinephrine, serotonin, histamine, epinephrine, Gamma-aminobutyric acid (GABA), glycine, glutamate, adenosine, inosine and aspartate.

Neurotransmitter analog agents include neurotransmitter agonists and antagonists. Examples of neurotransmitter agonists that can be used in the present invention include, but are not limited to almotriptan, aniracetam, atomoxetine, benserazide, bromocriptine, bupropion, cabergoline, citalopram, clomipramine, desipramine, diazepam, dihydroergotamine, doxepin duloxetine, eletriptan, escitalopram, fluvoxamine, gabapentin, imipramine, moclobemide, naratriptan, nefazodone, nefiracetam acamprosate, nicergoline, nortryptiline, paroxetine, pergolide, pramipexole, rizatriptan, ropinirole, sertraline, sibutramine, sumatriptan, tiagabine, trazodone, venlafaxine, and zolmitriptan.

Examples of neurotransmitter antagonist agents that can be used in the present invention include, but are not limited to 6 hydroxydopamine, phentolamine, rauwolfa alkaloid, eticlopride, sulpiride, atropine, promazine, scopolamine, galanin, chlorpheniramine, cyproheptadine, dihenylhydramine, methylsergide, olanzapine, citalopram, fluoxetine, fluoxamine, ketanserin, oridanzetron, p chlophenylalanine, paroxetine, sertraline and venlafaxine.

Particularly useful in the present invention are therapeutic agents such as peptides (e.g., neuropeptides) which have specific effects in the body but which under normal conditions poorly penetrate a cell membrane or blood barrier. In addition bacteria (e.g. gram negative bacteria) may build up resistance to antibiotics such as aminoglycosides, β lactams and quinolones by making their cell membrane less permeable. Addition of the carrier composition of the present invention may increase in vivo uptake into these bacteria, thereby enhancing the effectivity of the antibiotic therapeutic agent. Another example where the carrier composition of the present invention may be particularly useful is together with chelation agents such as EDTA for the treatment of high blood pressure, heart failure and atherosclerosis. The chelation agent is responsible for removing Calcium from arterial plaques. However, the arterial cellular membranes are relatively impermeable to chelating agents. Thus by incorporating the carrier composition of the present invention together with chelating agents, their bioavailability would be greatly enhanced.

The term “neuropeptides” as used herein, includes peptide hormones, peptide growth factors and other peptides. Examples of neuropeptides which can be used in accordance with the present invention include, but are not limited to Oxytocin, Vasopressin, Corticotropin releasing hormone (CRH), Growth hormone releasing hormone (GHRH), Luteinizing hormone releasing hormone (LHRH), Somatostatin growth hormone release inhibiting hormone, Thyrotropin releasing hormone (TRH), Neurokinin a (substance K), Neurokinin β, Neuropeptide K, Substance P, β-endorphin, Dynorphin, Met- and leu-enkephalin, Neuropeptide tyrosine (NPY), Pancreatic polypeptide, Peptide tyrosine-tyrosine (PYY), Glucogen-like peptide-1 (GLP-1), Peptide histidine isoleucine (PHI), Pituitary adenylate cyclase activating peptide (PACAP), Vasoactive intestinal polypeptide (VIP), Brain natriuretic peptide, Calcitonin gene-related peptide (CGRP) (α- and β-form), Cholecystokinin (CCK), Galanin, Islet amyloid polypeptide (IAPP), Melanin concentrating hormone (MCH), Melanocortins (ACTH, α-MSH and others), Neuropeptide FF, Neurotensin, Parathyroid hormone related protein, Agouti gene-related protein (AGRP), Cocaine and amphetamine regulated transcript (CART)/peptide, Endomorphin-1 and -2,5-HT-moduline, Hypocretins/orexins Nociceptin/orphanin FQ, Nocistatin, Prolactin releasing peptide, Secretoneurin and Urocortin

As mentioned, the present invention may be used to enhance in vivo delivery of diagnostic agents. Examples of diagnostic agents which can be used in accordance with the present invention include the x-ray imaging agents, fluorescent imaging agents and contrast media. Examples of x-ray imaging agents include WIN-8883 (ethyl 3,5-diacetamido-2,4,6-triiodobenzoate) also known as the ethyl ester of diatrazoic acid (EEDA), WIN 67722, i.e., (6-ethoxy-6-oxohexyl-3,5-bis(ace-tamido)-2,4,6-triiodobenzoate; ethyl-2-(3,5-bis(acetamido)-2,4,6-triiodo-b-enzoyloxy) butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy acetamide (WIN 65312); isopropyl 2-(3,5-bis(acetamido)-2,4,6-triiodobenzoyloxy) acetamide (WIN 12855); diethyl 2-(3,5-bis(acetamido)-2,4,6-triiodobenzoyl-oxy malonate (WIN 67721); ethyl 2-(3,5-bis(acetamido)-2,4,6-triiodobenzoyl-oxy) phenylacetate (WIN 67585); propandioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate) ester (WIN 68209). Other contrast media include, but are not limited to, magnetic resonance imaging aids such as gadolinium chelates, or other paramagnetic contrast agents. Examples of such compounds are gadopentetate dimeglumine (Magnevist®) and gadoteridol (Prohance®). Patent Application No. 20010001279 describes liposome comprising microbubbles which can be used as ultrasound contrast agents. Thus, diagnostic contrast agents can also be used in corporation with the present invention for aiding in ultrasound imaging of the brain.

Labeled antibodies may also be used as diagnostic agents in accordance with this aspect of the present invention. Use of labeled antibodies is particularly important for diagnosing diseases such as Alzheimer's where presence of specific proteins (e.g., β amyloid protein) are indicative of the disease.

A description of classes of therapeutic agents and diagnostic agents and a listing of species within each class can be found in Martindale, The Extra Pharmacopoeia, Twenty ninth Edition, The Pharmaceutical Press, London, 1989 which is incorporated herein by reference and made a part hereof. The therapeutic agents and diagnostic agents are commercially available and/or can be prepared by techniques known in the art.

As mentioned above, the carrier composition may also be used to enhance the penetration of a cosmetic agent. A cosmetic agent of the present invention can be, for example, an anti-wrinkling agent, an anti-acne agent, a vitamin, a skin peel agent, a hair follicle stimulating agent or a hair follicle suppressing agent. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid, collagen and many other agents which are known in the art.

The pharmaceutical agent of the present invention may be selected to treat or diagnose any pathology or condition. Pharmaceutical compositions of the present invention may be particularly advantageous to those tissues protected by physical barriers. For example, the skin is protected by an outer layer of epidermis. This is a complex structure of compact keratinized cell remnants (tough protein-based structures) separated by lipid domains. Compared to the oral or gastric mucosa, the stratum corneum is much less permeable to molecules either external or internal to the body.

Examples of skin pathologies which may be treated or diagnosed by the pharmaceutical compositions of the present invention include, but are not limited to acne, psoriasis, vitiligo, a keloid, a burn, a scar, a wrinkle, xerosis, ichthyosis, keratosis, keratoderma, dermatitis, pruritis, eczema, skin cancer, a hemorrhoid and a callus.

The pharmaceutical agent of the present invention may be selected to treat a tissue which is protected by a blood barrier (e.g. the brain). Examples of brain conditions which may be treated or diagnosed by the agents of the present invention include, but are not limited to brain tumor, neuropathy, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

The pharmaceutical composition of the present invention may also comprise other physiologically acceptable carriers (i.e., in addition to the above-described carrier composition) and excipients which will improve administration of a compound to the individual.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions of the present invention may be administered to an individual (e.g. mammal such as a human) using various routes of administration. Examples of routes of administration include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes. Manufacturing of the nanostructures and liquid is described hereinabove.

Pharmaceutical compositions for use in accordance with the present invention thus may be formulated in conventional manner using the carrier composition of the present invention either in the presence or absence of other physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in the carrier composition of the present invention, preferably in the presence of physiologically compatible buffers such as Hank's solution, Ringer's solution, or physiological salt buffer. For transmucosal administration, other penetrants appropriate to the barrier to be permeated may be used in the formulation. Such penetrants are generally known in the art.

For oral administration, the pharmaceutical composition can be formulated readily by combining the active compounds with the carrier composition of the present invention. The carrier composition preferably enables the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

For parenteral administration, the active ingredients may be combined with the carrier composition of the present invention either in the presence or absence of other solvents. Aqueous injection suspensions may contain substances, which increase the viscosity of the suspension, such as sodium carboxymethyl cellulose, sorbitol or dextran. Optionally, the suspension may also contain suitable stabilizers or other agents which increase the solubility of the active ingredients to allow for the preparation of highly concentrated solutions.

The pharmaceutical compositions of the present invention may be formulated for topical administration. Examples of topical formulations include, but are not limited to a gel, a cream, an ointment, a paste, a lotion, a milk, a suspension, an aerosol, a spray, a foam and a serum.

Alternatively, the active ingredient may be in powder form for constitution with the carrier composition of the present invention, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Effect of the Carrier Composition on Transformation Efficiencies in Electrocompetent Cells

Materials and Methods

Preparation of Electrocompetent Cells: Electro-Competent Cells were Prepared according to a standard protocol in which the water component (H₂O) was substituted with the carrier composition (Neowater™—Do-Coop technologies, Israel) at different steps and in different combinations. E. Coli cells were grown in rich media until the logarithmic phase and then harvested by centrifugation. This rich media has a rich nutrient base which provides amino acids, vitamins, inorganic and trace minerals at levels higher than those of LB Broth. The medium is buffered at pH 7.2±0.2 with potassium phosphate to prevent a drop in pH-and to provide a source of phosphate. These modifications permit higher cell yields than can be achieved with LB. The pellets were washed three times in standard cold water and re-suspended in either water containing 10% glycerol (standard) or in the carrier composition containing 2, 5, or 10% glycerol and frozen at −80° C. Electroporation was performed under standard conditions using pUC plasmid DNA diluted in water and the bacteria was plated on LB plates comprising antibiotic to for colony counting. Colonies were counted the following day and transformation efficiency was determined.

Results

As illustrated in FIG. 1, resuspension of electrocompetent bacteria in all dilutions of the carrier composition increases transformation efficiencies in all cases (from 10 to 17 fold).

### Example 2

### Effect of Liquid and Nanostructures on DNA Uptake in Chemically Competent Cells

The effect of the carrier composition on DNA uptake by different chemically competent cells was studied.

Methods

Bacterial strains: XL1-Blue

pUC plasmid DNA was diluted 1:10 in either water or the carrier composition (Neowater™—Do-Coop technologies, Israel) and was used for transformation of three bacteria strains, using the heat shock method. Essentially, following incubation for ten minutes on ice, the DNA together with the bacteria were incubated at 42° C. for 30 seconds and plated on LB plates comprising antibiotic for colony counting. Colonies were counted the following day and transformation efficiency was determined.

Results

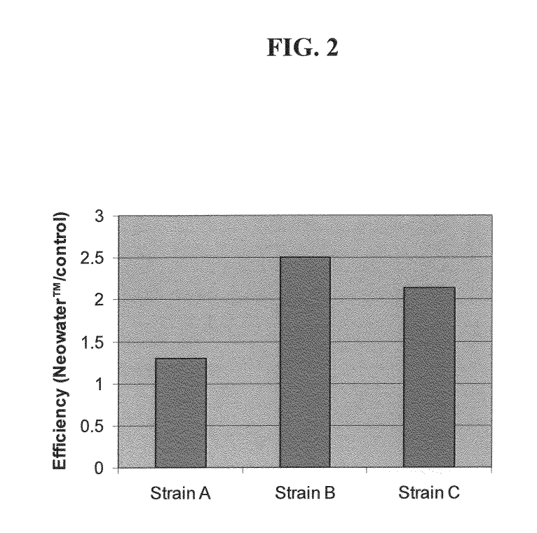

As depicted in FIG. 2, dilution of DNA in the carrier composition significantly improved DNA uptake by competent cells by 30-150%, varying according to the bacterial strain.

### Example 3

### Effect of the Carrier Composition on DNA Uptake in a Primary Human Cell Culture

Materials and Methods

Cell culture: Human bone marrow primary cells were grown in Mem-alpha 20% fetal calf serum and plated so that they were 80% confluent 24 hours prior to cell culture.

Transfection: Cells were transfected using a standard Lipofectamine 2000 (Invitrogen™) transfection procedure following the manufacturer protocol with a green fluorescent protein (GFP) construct. The transfection was repeated using a mix of the carrier composition (Neowater™—Do-Coop technologies, Israel) and 12.5% of the amount of Lipofectamine 2000 used in the control experiment.

Results

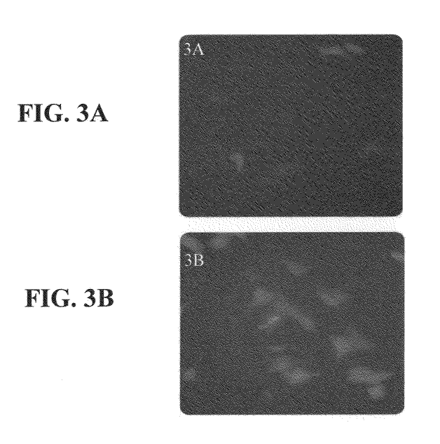

As can be seen from FIGS. 3A-B, transfection efficiency in primary cells was increased using the carrier composition together with Lipofectamine 2000.

### Example 4

### Effect of the Carrier Composition on Phage-Bacteria Interaction

Methods

Phage typing: Two specific international phage strains (#6 and #83A) of Staphylococcus aureus, and all culture media were obtained from Public Health Laboratory in Colindale, UK. Assay conditions and procedures were performed according to standard protocols. Each bacteriophage was tested at 1 and 100 RTD (Routine Test Dilution) and propagated in parallel in water—or the carrier composition—(Neowater™—Do-Coop technologies, Israel) based agar plates (of 2 different lots). Statistical analysis was performed by using 2 ways ANOVA using SPSS.

Infection of the host bacterial strain by the phage: Competent E. coli XL1 Blue MRA (Stratagene) cells were prepared using standard protocols. Phage λ GEM 11 (Promega) suspensions were prepared from phage stock in SM buffer in series of 1/10 dilutions either based on the carrier composition or ddH₂O. 1 μl of each dilution was incubated with 200 μl of competent bacterial host E. coli XL1 Blue MRA. The mix was incubated at 37° C. for 15 min to allow the bacteriophage to inject its DNA into the host bacteria. After incubation a hot (45-50° C.) top agarose was added and the suspension was dispersed on the LB plate. Nine replications of each dilution and treatment were prepared. The PFU (plaque forming unit) were counted following overnight incubation.

Results

Phage infectivity: The effect of the carrier composition on phage infectivity was tested by infecting bacteria with a specific phage strain at limiting dilutions (100×) of RTD, and examining plaque formation on either the carrier composition or control agar plates. As shown in FIGS. 4A-B, plaques were formed in the first two serial dilutions. However, in dilution #3 a plaque was present on the carrier composition plate but not in the control counterpart, representing a 100 fold increase in infectivity.

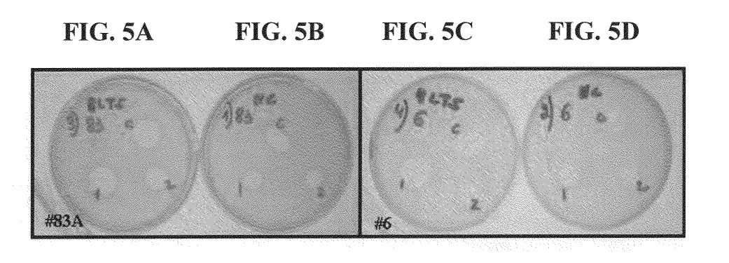

Time of plaque formation and plaque size: The kinetics of the bacteria-bacteriophage reaction was measured. Specific phage strains were used for infection of S. aureus plated onto either control or carrier composition soft agar plates at 1 or 100RTD and incubated at 37° C. Within 1 hour of incubation plaques were observed in the carrier composition but not in the control plates. Three hours later plaques were visible also in the control plates (FIGS. 5A-D) but remained significantly smaller than those observed in carrier composition plates (p=0.014 by 2-ways ANOVA).

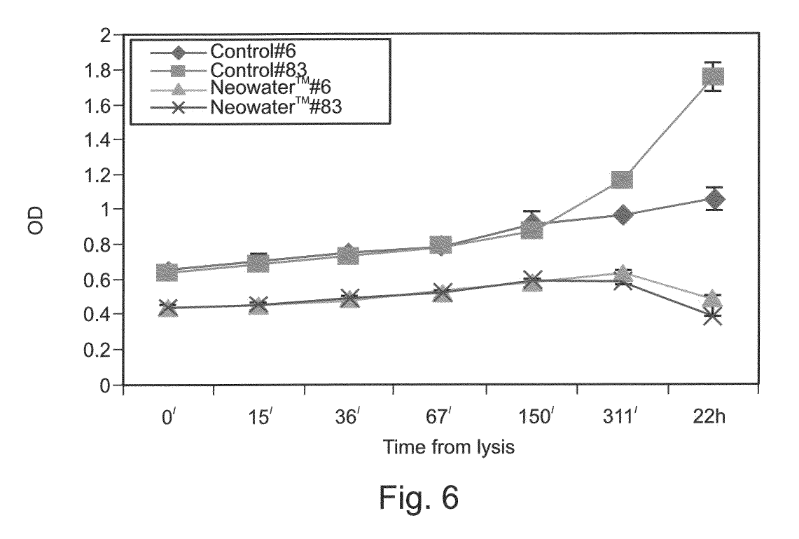

Bacterial Lysis: As illustrated in FIG. 6, lysis was significantly improved (more than 30%) in carrier composition-based growth media compared to control (p=0.001 by 2-ways ANOVA), and remained as such for more than 5 hours. Following 22 hours in culture, a second lysis burst was noticed in the carrier composition growth media while in control the culture became cloudy due to bacteria overgrowth.

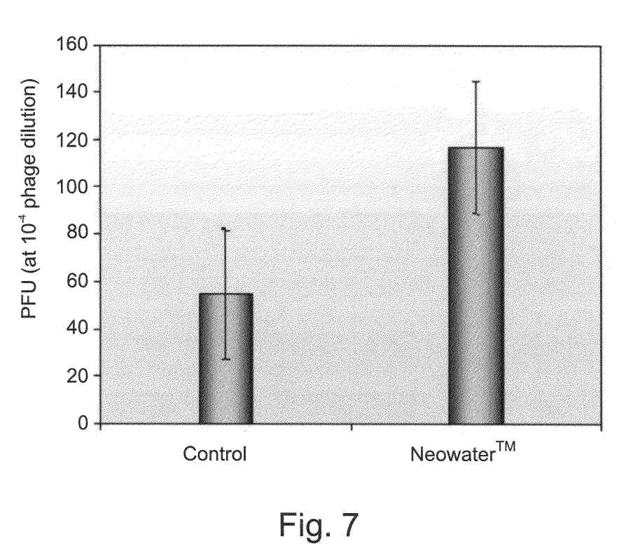

Phage λ GEM 11 PFU in E. coli XL1 Blue bacteria: Phage (λ GEM 11) suspensions were prepared from phage stock in either control or carrier composition-based SM buffer in series of 1/10 dilutions, mixed with the competent bacterial host and plated on agar plates at either 10⁻³ or 10⁻⁴ phage dilution. The PFUs were counted following overnight incubation. As shown in FIG. 7, a significant increase in the phage titer was observed in carrier composition-diluted phage samples, at 10⁻⁴ phage dilution (2 folds; p=0.01). The effect at lower dilutions (i.e. more concentrated phage suspension) was lower and was not statistically significant. Nine replications of each dilution and treatment were prepared. The pfu were counted after overnight incubation at 10⁻⁴ phage dilution.

Conclusions

The carrier composition facilitates a significant decrease in RTD (up to 100 fold) and better phage infectivity, as well as generation of additional lysis cycle after 22 hours in liquid culture.

The kinetics of phage-host interaction is significantly enhanced in the carrier composition containing growth media as observed by accelerated burst time and larger plaque size compared to the control media.

At low phage concentrations the carrier composition increases PFU titer over standard solutions

Taken together, it may be suggested that the carrier composition is mostly significant in the absorption step enabling a better DNA uptake by the bacteria hence increasing transduction efficiency.

### Example 5

### Effect of the Carrier Composition on Colony Uptake of Antibiotic

Bacterial colonies were grown on peptone/agar plates in the presence and absence of antibiotic. The effect of the carrier composition on colony uptake of antibiotic was ascertained.

Materials and Methods

Colony growth: Bacillus subtilis bacterial colonies were pre-grown in the presence and absence of the carrier composition (Neowater™—Do-Coop technologies, Israel) and subsequently plated on 0.5% agar with 10 g/l peptone. 10̂5 bacterial colonies were pre-grown in the presence and absence of the carrier composition (Neowater™—Do-Coop technologies, Israel) and in the presence of SP water (reverse osmosis-water mixed with the same source powder as in Neowater™) and subsequently plated on 0.5% agar with 10 g/l peptone. T strain bacterial colonies were pre-grown in the presence and absence of the carrier composition (Neowater™—Do-Coop technologies, Israel) and subsequently plated on 1.75% agar with 5 g/l peptone (prepared using the liquid composition of the present invention) both in the presence and absence of streptomycin at the same minimum inhibitory concentration (MIC).

Results

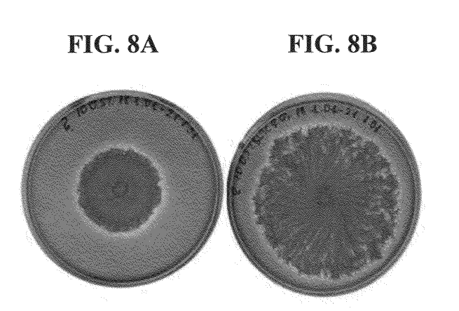

As illustrated in FIGS. 8A and 8B, the bacterial colony was larger in the presence of the carrier composition. The colony also showed a different pattern in the presence of the carrier composition, with branches being more separate compared to control plates.

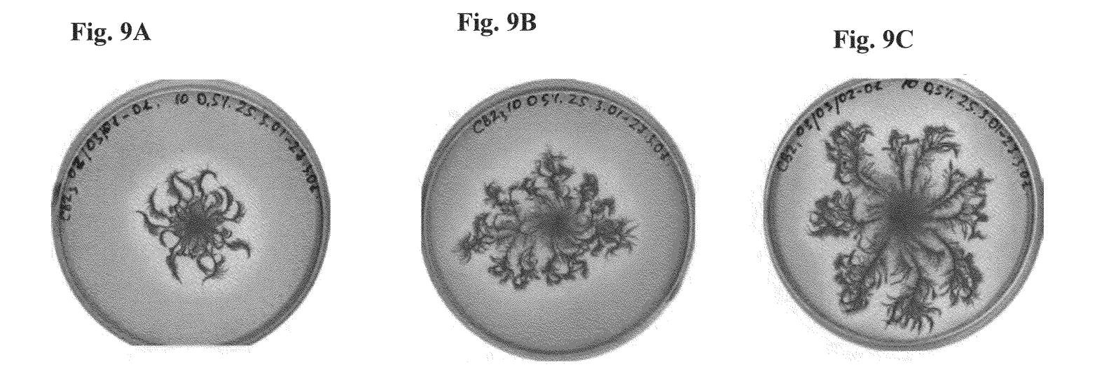

As illustrated in FIGS. 9A-C, the carrier composition leads to faster bacterial growth relative to reverse osmosis-water while SP water exhibits slower growth.

Following streptomycin antibiotic to the substrate, the colonies were smaller (FIGS. 10A and 10B). When both streptomycin and the carrier composition were added to the substrate, the colony pattern changed and the colony size diminished considerably (FIG. 10C).

### Example 6

### Effect of the Carrier Composition on Growth and Photo-Luminescence of Bacteria

Methods

Bioluminescent Vibrio Harveyi bacteria (BB120 strain) were grown in either medium comprising the carrier composition (Neowater™—Do-Coop technologies, Israel) or medium comprising distilled water. Luminescent measurements were made using an ELISA reader, Model: Spectrafluor+, MFR: Tecan at defined intervals. Turbidity was measured by same equipment

Results

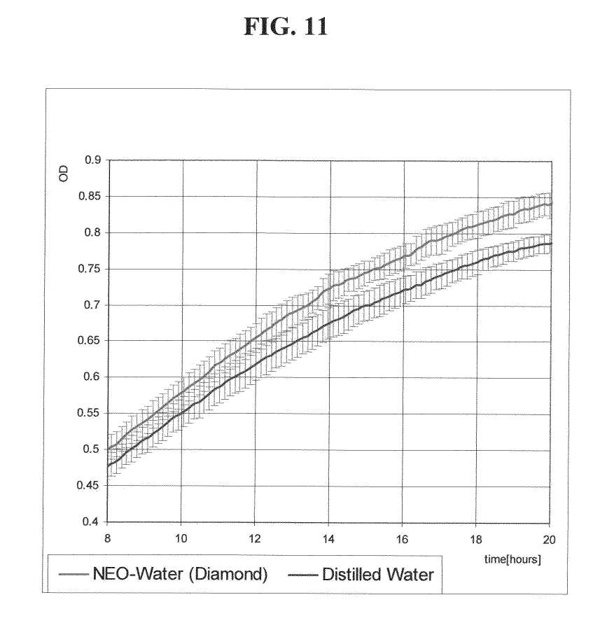

Turbidity values taken from the 15^{th} hour indicate that the average growth in bacteria pre-grown in medium comprising the carrier composition is 6.5%±2.75 higher then the average growth of bacteria pre-grown in distilled water medium (FIG. 11).

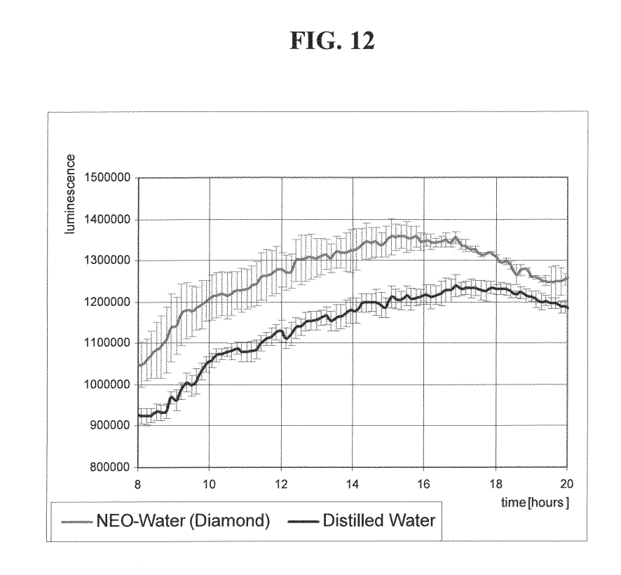

As illustrated in FIG. 12, luminescence values taken from the 15^{th} hour illustrate that the average luminescence in bacteria pre-grown in medium comprising the carrier composition is 9.97%±2.27 higher then the luminescence of bacteria pre-grown in distilled water medium.

Conclusion

The results indicate that the carrier composition increases the growth of Vibrio bacteria and also increases the expression of the luminescence gene.

### Example 7

### Effect of the Carrier Composition on Commercial Skin Cream Uptake In-Vivo

Patients suffering from acne were topically administered with a commercial skin cream in the presence and absence of the carrier composition (Neowater™—Do-Coop technologies, Israel). The therapeutic benefit of the carrier composition to the skin cream was measured by UV light Facial Stage, Moritex, Japan.

Materials and Methods

Skin cream: A commercial skin cream Clearasil, Alleon Pharmacy was prepared in the presence of the carrier composition at a dilution of 1:1.

Patient criteria: severe case of facial acne.

Treatment regimen: The skin cream was applied to patients once a day for three days

Measurement of skin improvement: Skin improvement was measured by UV light Facial Stage, Moritex, Japan

Results

As illustrated in FIGS. 13A-C, the number of patient spots declined rapidly over a period of three days (from 229 spots to 18 spots), following treatment with the commercial skin cream in the presence of the carrier composition. In the absence of the carrier composition, the number of spots declined from 229 to 18.

### Example 8

### Ultrasonic Tests

The carrier composition of the present invention was subjected to a series of ultrasonic tests in an ultrasonic resonator.

Materials and Methods

Measurements of ultrasonic velocities in the carrier composition of the present invention (referred to in the present Example as Neowater™) and double distilled (dist.) water were performed using a ResoScan® research system (Heidelberg, Germany).

Calibration: Both cells of the ResoScan® research system were filled with standard water (demin. Water Roth. Art.3175.2 Charge:03569036) supplemented with 0.005% Tween 20 and measured during an isothermal measurement at 20° C. The difference in ultrasonic velocity between both cells was used as the zero value in the isothermal measurements as further detailed hereinbelow.

Isothermal Measurements: Cell 1 of the ResoScan® research system was used as reference and was filled with dist. Water (Roth Art. 34781 lot#48362077). Cell 2 was filled with the carrier composition of the present invention. Absolute Ultrasonic velocities were measured at 20° C. In order to allow comparison of the experimental values, the ultrasonic velocities were corrected to 20.000° C.

Results

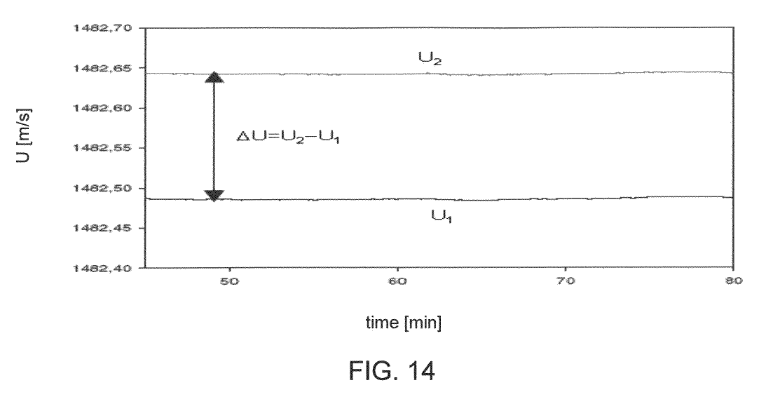

FIG. 14 shows the absolute ultrasonic velocity U as a function of observation time, as measured at 20.051° C. for the carrier composition of the present invention (U₂) and the dist. water (U₁). Both samples displayed stable isothermal velocities in the time window of observation (35 min).

Table 1 below summarizes the measured ultrasonic velocities U₁, U₂ and their correction to 20° C. The correction was calculated using a temperature-velocity correlation of 3 m/s per degree centigrade for the dist. Water.

TABLE 1

| | | |
|---|---|---|
| Sample | Temp | U |

| dist. water | 20.051° | C. | 1482.4851 |
|---|---|---|---|
| Neowater ™ | | | 1482.6419 |
| dist. water | 20° | C. | 1482.6381 |

| | |
|---|---|
| Neowater ™ | 1482.7949 |

As shown in FIG. 14 and Table 1, differences between dist. water and the carrier composition of the present invention were observed by isothermal measurements. The difference ΔU=U₂−U₁ was 15.68 cm/s at a temperature of 20.051° C. and 13.61 cm/s at a temperature of 20° C. The value of ΔU is significantly higher than any noise signal of the ResoScan® system. The results were reproduced on a second ResoScan® research system.

### Example 9

### Hydrophobic Properties of the Carrier Composition of the Present Invention

The carrier composition of the present invention was subjected to a series of tests in order to determine if it comprised hydrophobic properties.

Materials and Experimental Methods

Materials: Neowater™ (Do-Coop technologies, Israel); coloring agent Phenol Bromide Blue (Sigma-Aldrich).

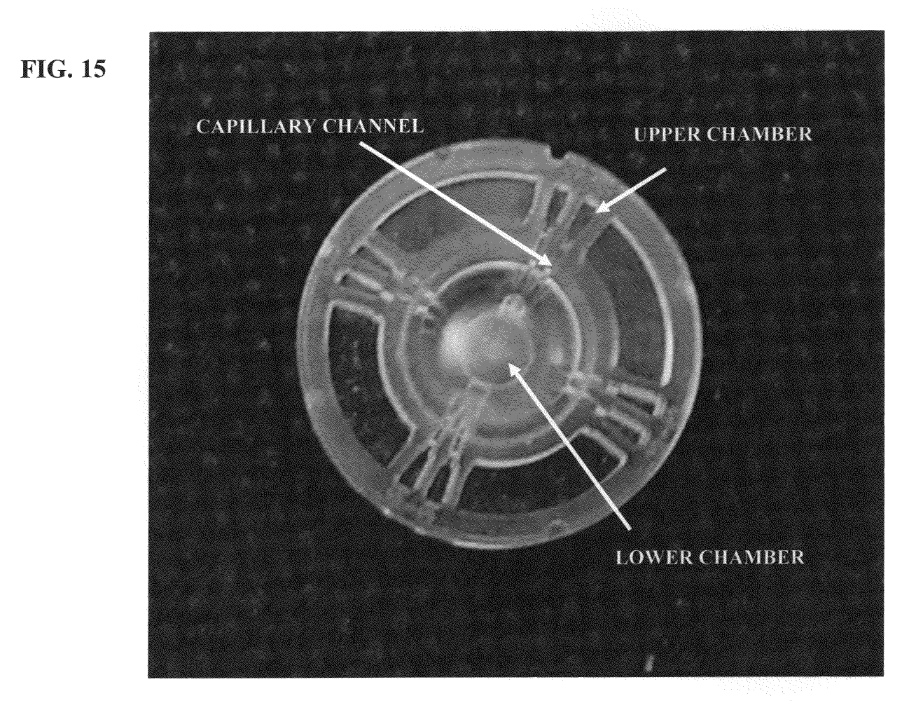

Plastic apparatus: An apparatus was constructed comprising an upper and lower chamber made from a hydrophobic plastic resin (proprietary resin, manufactured by MicroWebFab, Germany). The upper and lower chambers were moulded such that very narrow channels which act as hydrophobic capillary channels interconnect the four upper chambers with the single lower chamber. These hydrophobic capillary channels simulate a typical membrane or other biological barriers (FIG. 15).

Method: The color mix was diluted with the liquid composition of the present invention or with water at a 1:1 dilution. A ten microlitre drop of the liquid composition of the present invention+color composition was placed in the four upper chambers of a first plastic apparatus, whilst in parallel a five hundred microlitre drop of the liquid composition of the present invention was placed in the lower chamber directly above the upper chambers. Similarly a ten microlitre drop of water+color composition was placed in the four upper chambers, of a second plastic apparatus whilst in parallel a five hundred microlitre drop of water was placed in the lower chamber directly above the upper chambers. The location of the dye in each plastic apparatus was analyzed fifteen minutes following placement of the drops.

Results

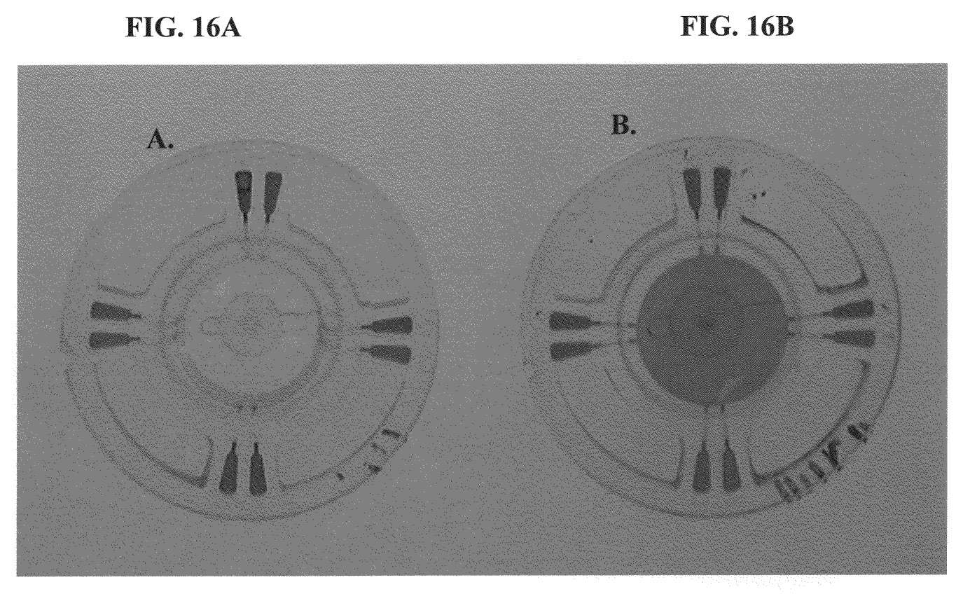

The lower chamber of the plastic apparatus comprising the Water and color mix is clear (FIG. 16A), while the lower chamber of the plastic apparatus comprising the liquid composition of the present invention and color mix, exhibits a light blue color (FIG. 16B).

Conclusion

The liquid composition of the present invention comprises hydrophobic properties as it is able to flow through a hydrophobic capillary.

### Example 10

### Buffering Capacity of the Carrier Composition

The effect of the carrier composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800nm, but the graph refers to a wavelength of 557 nm alone, in relation to addition of 0.02M Sodium hydroxide.

Results

Table 2 summarizes the absorbance at 557 nm of each water solution following sodium hydroxide titration.

TABLE 2

| | | | | | | | μl of 0.02 |
|---|---|---|---|---|---|---|---|
| | | | | | | | M sodium |
| NW 1 | NW 2 | NW 3 | NW 4 | NW 5 | | | hydroxide |
| HAP | AB 1-2-3 | HA 18 | Alexander | HA-99-X | NW 6 | RO | added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

As illustrated in FIG. 17 and Table 2, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater™.

In summary, from this experiment, all new water types comprising nanostructures tested (HAP, AB 1-2-3, HA-18, Alexander) shows similar characters to Neowater™, except HA-99-X.

### Example 11

### Buffering Capacity of the Carrier Comprising Nanostructures

The effect of the carrier composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration:—1 μl to 15 μl of 1M sodium hydroxide (Sodium hydroxide) was added.

Hydrochloric acid titration:—1 μl to 15 μl of 1M Hydrochloric acid was added.

Results

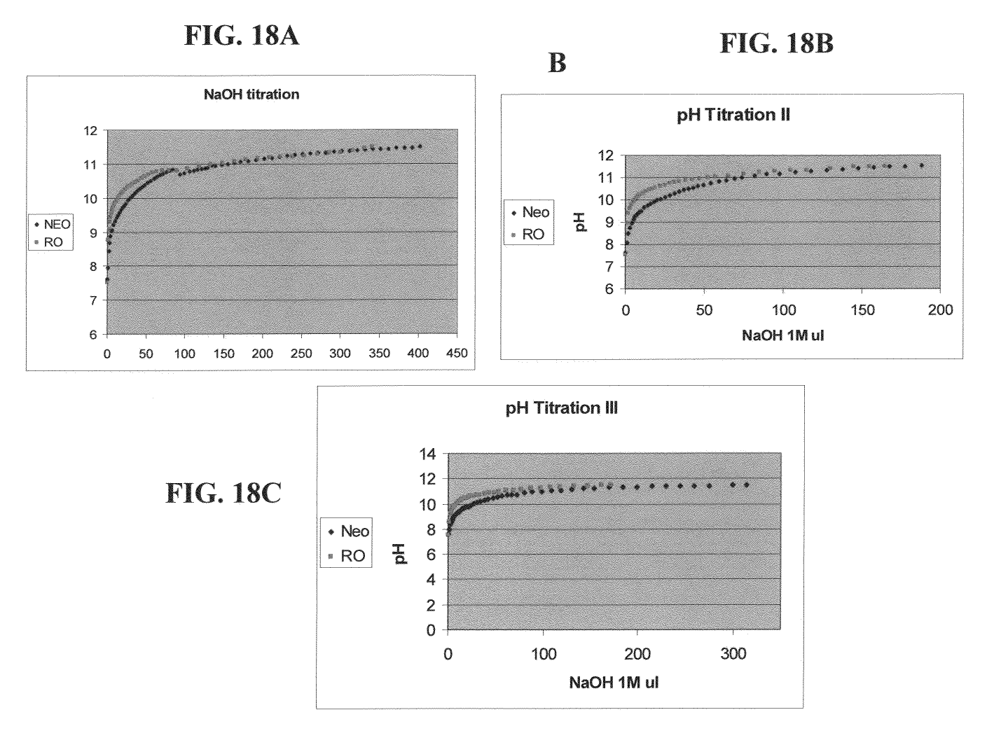

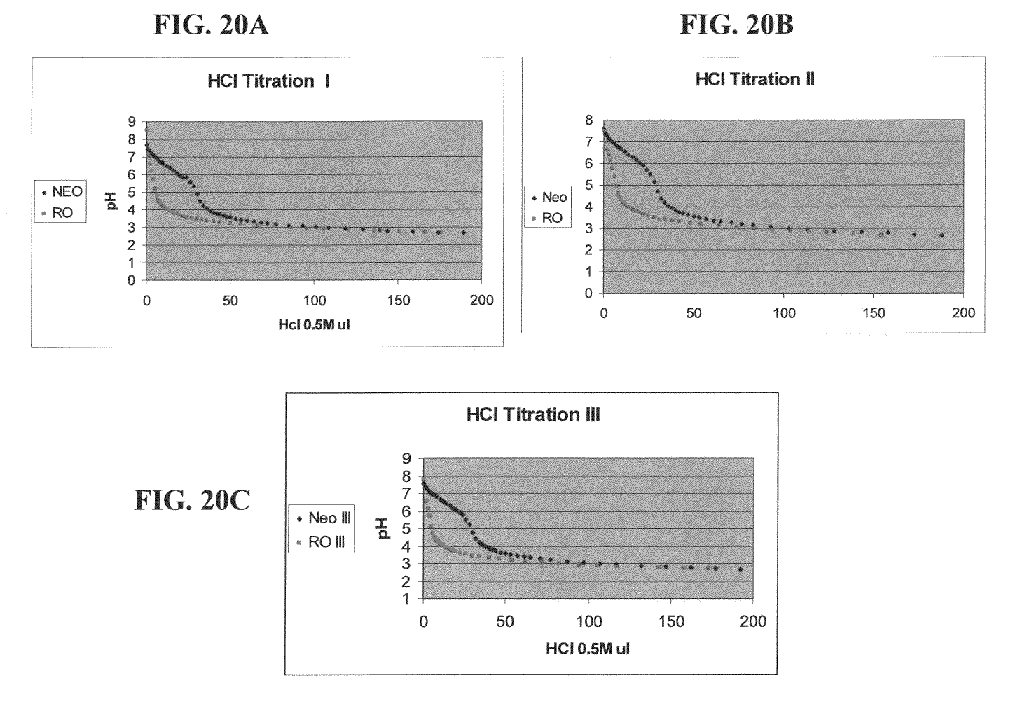

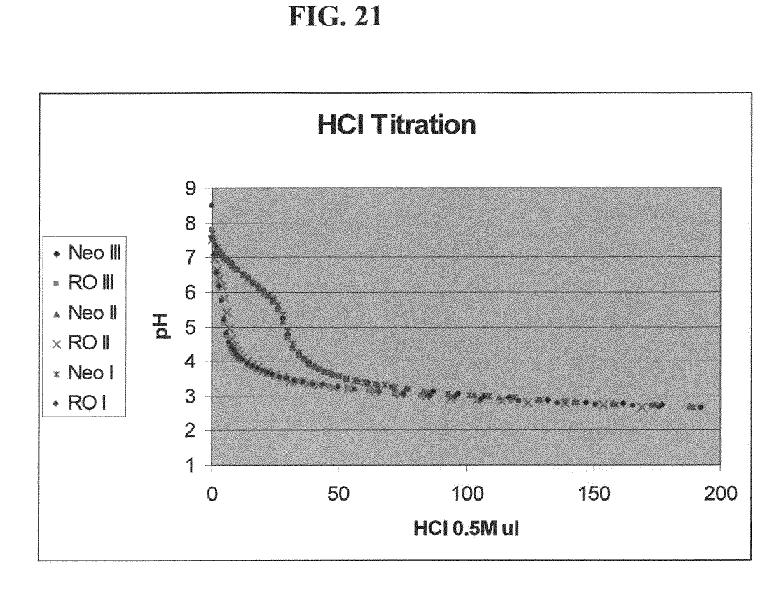

The results for the sodium hydroxide titration are illustrated in FIGS. 18A-C and 19A-C. The results for the Hydrochloric acid titration are illustrated in FIGS. 20A-C and FIG. 21.

The water comprising nanostructures has buffering capacities since it requires greater amounts of sodium hydroxide in order to reach the same pH level that is needed for RO water. This characterization is more significant in the pH range of -7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl sodium hydroxide 1M (in a total sum) the pH of RO increased from 7.56 to 10.3. The pH of the water comprising nanostructures increased from 7.62 to 9.33. When adding 10 μl Hydrochloric acid 0.5M (in a total sum) the pH of RO decreased from 7.52 to 4.31 The pH of water comprising nanostructures decreased from 7.71 to 6.65. This characterization is more significant in the pH range of—7.7-3.

### Example 12

### Buffering Capacity of the Carrier Comprising Nanostructures

The effect of the carrier composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric Acid Titration:

### RO: 45 ml pH 5.8

1 ml phenol red and 5 μl Sodium hydroxide 1M was added, new pH=7.85 Neowater™ (# 150905-106): 45 ml pH 6.3

1 ml phenol red and 4 μl Sodium hydroxide 1M was added, new pH=7.19

Sodium Hydroxide Titration:

### I. RO: 45 ml pH 5.78

1 ml phenol red, 6 μl Hydrochloric acid 0.25M and 4 μl Sodium hydroxide 0.5M was added, new pH=4.43

### Neowater™ (# 150604-109): 45 ml pH 8.8

1 ml phenol red and 45 μl Hydrochloric acid 0.25M was added, new pH=4.43

### II. RO: 45 ml pH 5.78

1 ml phenol red and 5 μl Sodium hydroxide 0.5M was added; new pH=6.46

### Neowater™ (# 120104-107): 45 ml pH 8.68

1 ml phenol red and 5 μl Hydrochloric acid 0.5M was added, new pH=6.91

Results

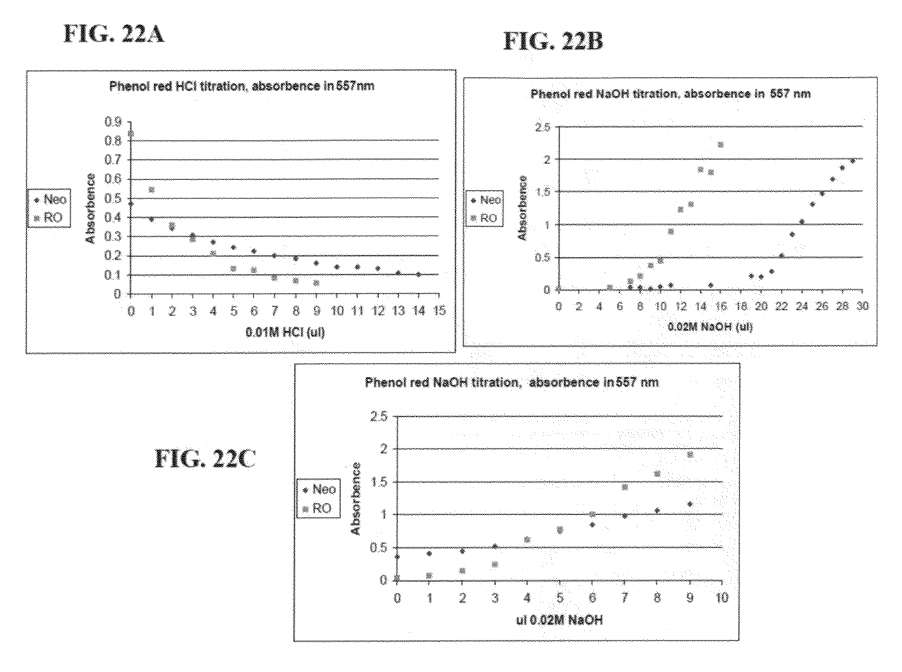

As illustrated in FIGS. 22A-C and 23A-B, the buffering capacity of water comprising nanostructures was higher than the buffering capacity of RO water.

### Example 13

### Buffering Capacity of RF Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl 0.5M Hydrochloric acid to 50 ml water. The titration was finished when the pH value reached below 4.2.

The experiment was performed in triplicates.

Results

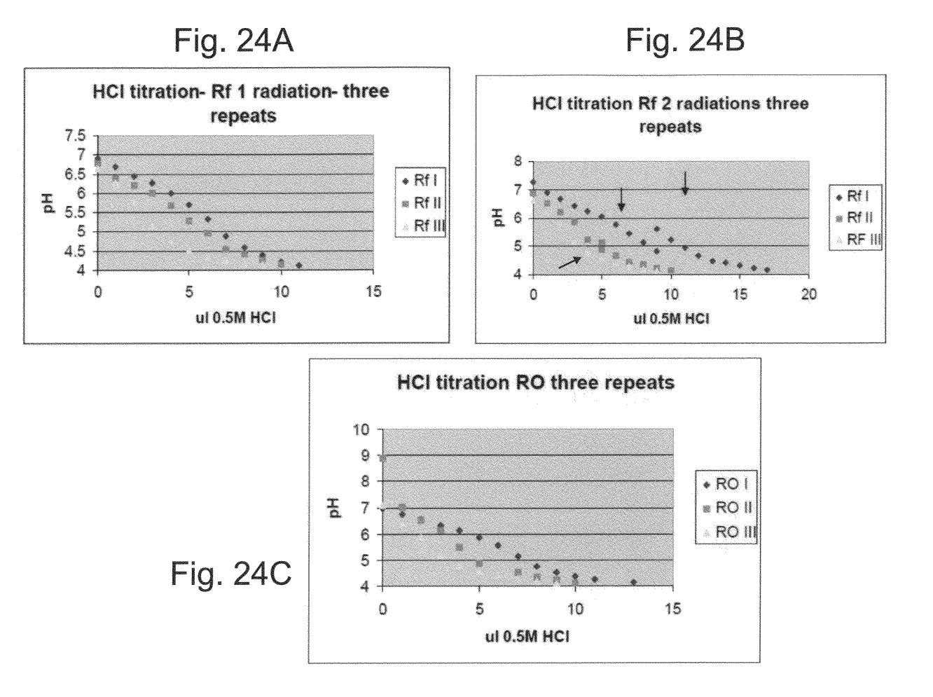

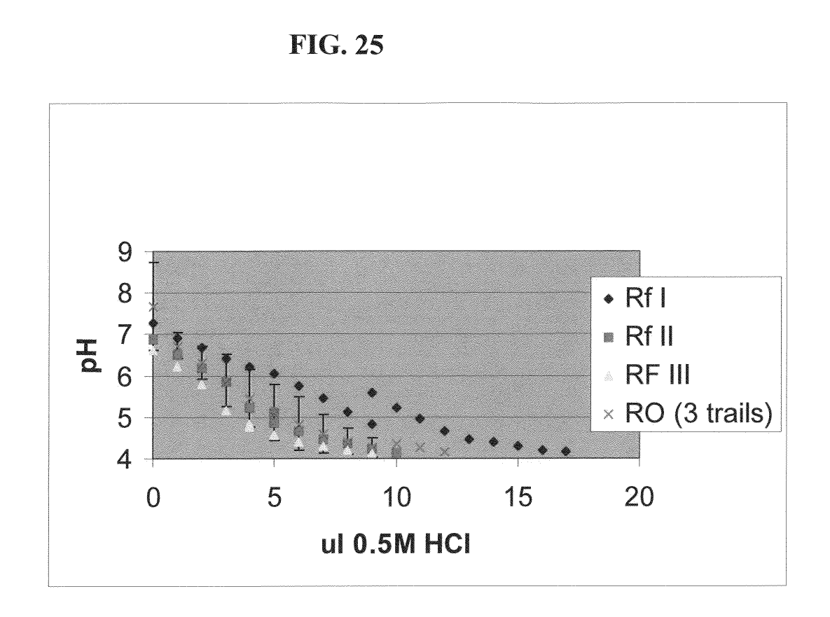

As can be seen from FIGS. 24A-C and FIG. 25, RF water and RF2 water comprise buffering properties similar to those of the carrier composition comprising nanostructures.

### Example 14

### Solvent Capability of the Carrier Comprising Nanostructures

The following experiments were performed in order to ascertain whether the carrier composition comprising nanostructures was capable of dissolving two materials both of which are known not to dissolve in water at a concentration of 1 mg/ml.

A. Dissolving in Ethanol/(Neowater™—Do-Coop Technologies, Israel) Based Solutions

Materials and Methods

Five attempts were made at dissolving the powders in various compositions. The compositions were as follows:

A. 10 mg powder (red/white)+990 μl Neowater™.
B. 10 mg powder (red/white)+990 μl Neowater™ (dehydrated for 90 min).
C. 10 mg powder (red/white)+495 μl Neowater™+495 μl EtOH (50%-50%).
D. 10 mg powder (red/white)+900 μl Neowater™+90 μl EtOH (90%-10%).
E. 10 mg powder (red/white)+820 μl Neowater™+170 μl EtOH (80%-20%).

The tubes were vortexed and heated to 60° C. for 1 hour.

Results

1. The white powder did not dissolve, in all five test tubes.

2. The red powder did dissolve however; it did sediment after a while.
It appeared as if test tube C dissolved the powder better because the color changed to slightly yellow.

B. Dissolving in Ethanol/(Neowater™—Do-Coop Technologies, Israel) Based Solutions Following Crushing

Materials and Methods

Following crushing, the red powder was dissolved in 4 compositions:

A. ½ mg red powder+49.5 μl RO.
B. ½ mg red powder+49.5 μl Neowater™
C. ½ mg red powder+9.9 μl EtOH→39.65 μl Neowater™ (20%-80%).
D. ½ mg red powder+24.75 μl EtOH→24.75 μl Neowater™ (50%-50%).
Total reaction volume: 50 μl.

The tubes were vortexed and heated to 60° C. for 1 hour.

Results

Following crushing only 20% of ethanol was required in combination with the Neowater™ to dissolve the red powder.

C. Dissolving in Ethanol/(Neowater™—Do-Coop Technologies, Israel) Solutions Following Extensive Crushing

Materials and Methods

Two crushing protocols were performed, the first on the powder alone (vial 1) and the second on the powder dispersed in 100 μl Neowater™ (1%) (vial 2).

The two compositions were placed in two vials on a stirrer to crush the material overnight:

15 hours later, 100 μl of Neowater™ was added to 1 mg of the red powder (vial no. 1) by titration of 10 μl every few minutes.

Changes were monitored by taking photographs of the test tubes between 0-24 hours (FIGS. 26F-J).

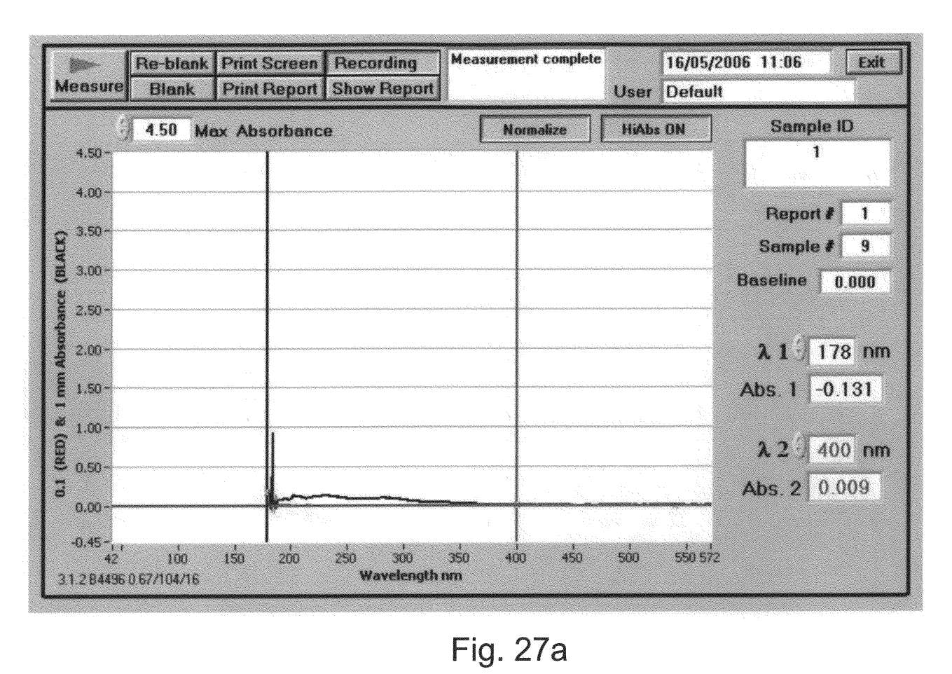

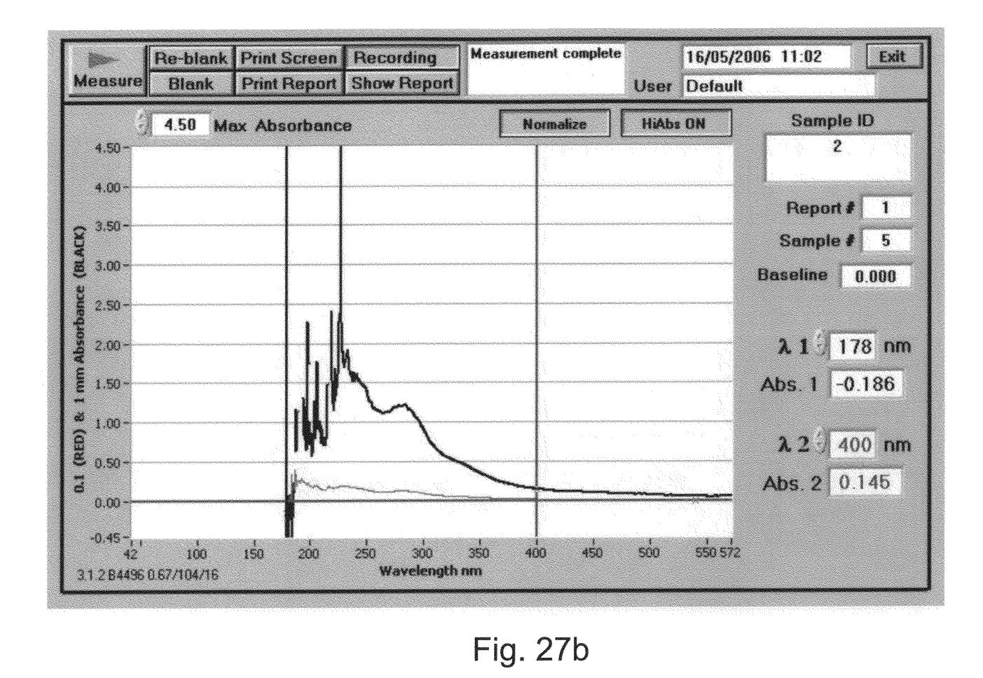

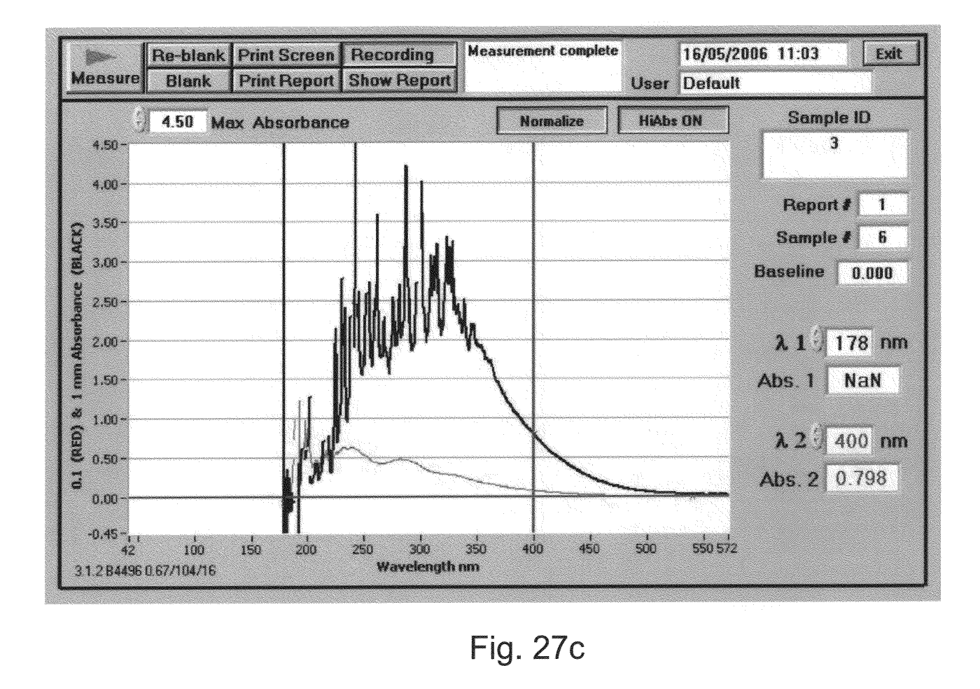

As a comparison, two tubes were observed one of which comprised the red powder dispersed in 990 μl Neowater™ (dehydrated for 90 min)—1% solution, the other dispersed in a solution comprising 50% ethanol/50% Neowater™)—1% solution. The tubes were heated at 60° C. for 1 hour. The tubes are illustrated in FIGS. 26A-E. Following the 24 hour period, 2 μl from each solution was taken and its absorbance was measured in a nanodrop (FIGS. 27A-C)

Results

FIGS. 26A-J illustrate that following extensive crushing, it is possible to dissolve the red material, as the material remains stable for 24 hours and does not sink. FIGS. 26A-E however, show the material changing color as time proceeds (not stable).

Vial 1 almost didn't absorb (FIG. 27A); solution B absorbance peak was between 220-270 nm (FIG. 27B) with a shift to the left (220 nm) and Solution C absorbance peak was between 250-330 nm (FIG. 27C).

Conclusions

Crushing the red material caused the material to disperse in Neowater™. The dispersion remained over 24 hours. Maintenance of the material in glass vials kept the solution stable 72 h later, both in 100% dehydrated Neowater™ and in EtOH-Neowater™ (50%-50%).

### Example 15

### Capability of the Carrier Comprising Nanostructures to Dissolve Daidzein, Daunrubicine and t-boc Derivative

The following experiments were performed in order to ascertain whether the carrier composition comprising nanostructures was capable of dissolving three materials—Daidzein-daunomycin conjugate (CD-Dau); Daunrubicine (Cerubidine hydrochloride); t-boc derivative of daidzein (tboc-Daid), all of which are known not to dissolve in water.

Materials and Methods

A. Solubilizing CD-Dau—Part 1:

Required concentration: 3 mg/ml Neowater.

Properties: The material dissolves in DMSO, acetone, acetonitrile.
Properties: The material dissolves in EtOH.

5 different glass vials were prepared:
1. 5 mg CD-Dau+1.2 ml Neowater™.
2. 1.8 mg CD-Dau+600 μl acetone.
3. 1.8 mg CD-Dau+150 μl acetone+450 μl Neowater™ (25% acetone).
4. 1.8 mg CD-Dau+600 μl 10%*PEG (Polyethylene Glycol).
5. 1.8 mg CD-Dau+600 μl acetone+600 μl Neowater™.

The samples were vortexed and spectrophotometer measurements were performed on vials #1, 4 and 5

The vials were left opened in order to evaporate the acetone (vials #2, 3, and 5).

Results

Vial #1 (100% Neowater): CD-Dau sedimented after a few hours.

Vial #2 (100% acetone): CD-Dau was suspended inside the acetone, although 48 hours later the material sedimented partially because the acetone dissolved the material.

Vial #3 (25% acetone): CD-Dau didn't dissolve very well and the material floated inside the solution (the solution appeared cloudy).

Vial #4 (10% PEG+Neowater): CD-Dau dissolved better than the CD-Dau in vial #1, however it didn't dissolve as well as with a mixture with 100% acetone.

Vial #5: CD-Dau was suspended first inside the acetone and after it dissolved completely Neowater™ was added in order to exchange the acetone. At first acetone dissolved the material in spite of Neowater™'s presence. However, as the acetone evaporated the material partially sediment to the bottom of the vial. (The material however remained suspended.

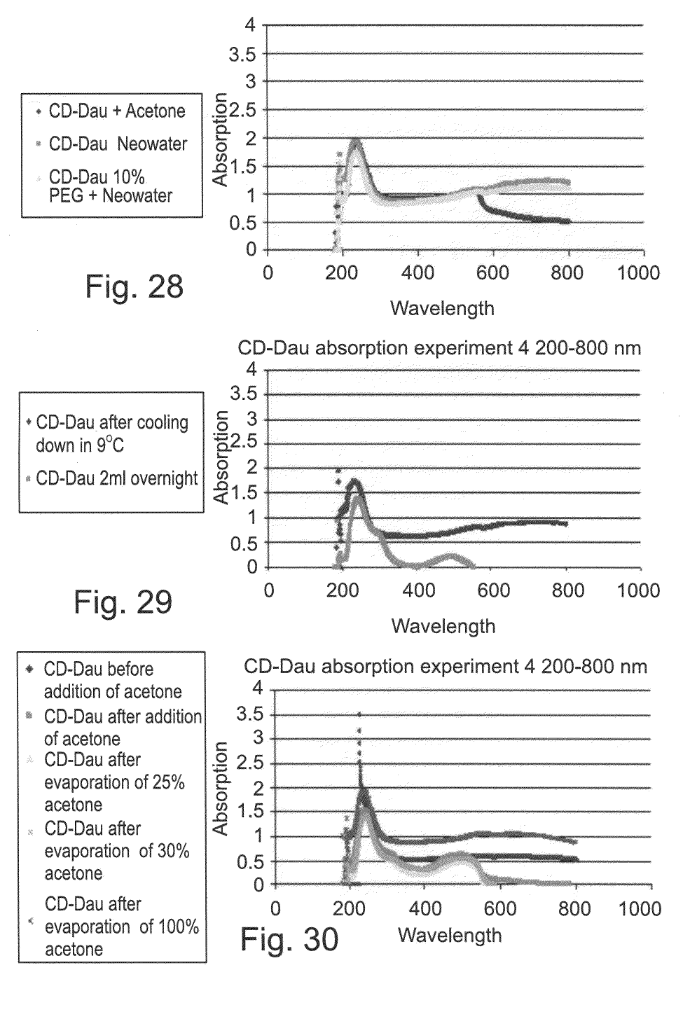

Spectrophotometer measurements (FIG. 28) illustrate the behavior of the material both in the presence and absence of acetone. With acetone there are two peaks in comparison to the material that is suspended with water or with 10% PEG, which in both cases display only one peak.

B. Solubilizing CD-Dau—Part 2:

As soon as the acetone was evaporated from solutions #2, 4 and 5, the material sedimented slightly and an additional amount of acetone was added to the vials. This protocol enables the dissolving of the material in the presence of acetone and Neowater™ while at the same time enabling the subsequent evaporation of acetone from the solution (this procedure was performed twice). Following the second cycle the liquid phase was removed from the vile and additional amount of acetone was added to the sediment material. Once the sediment material dissolved it was merged with the liquid phase removed previously. The merged solution was evaporated again. The solution from vial #1 was removed since the material did not dissolve at all and instead 1.2 ml of acetone was added to the sediment to dissolve the material. Later 1.2 ml of 10% PEG+Neowater™ were added also and after some time the acetone was evaporated from the solution. Finalizing these procedures, the vials were merged to one vial (total volume of 3 ml). On top of this final volume 3 ml of acetone were added in order to dissolve the material and to receive a lucid liquefied solution, which was then evaporated again at 50° C. The solution didn't reach equilibrium due to the fact that once reaching such status the solution would have been separated. By avoiding equilibrium, the material hydration status was maintained and kept as liquid. After the solvent evaporated the material was transferred to a clean vial and was closed under vacuum conditions.

C. Solubilizing CD-Dau—Part 3:

Another 3 ml of the material (total volume of 6 ml) was generated with the addition of 2 ml of acetone-dissolved material and 1 ml of the remaining material left from the previous experiments.

1.9 ml Neowater™ was added to the vial that contained acetone.

100 μl acetone+100 μl Neowater™ were added to the remaining material.

Evaporation was performed on a hot plate adjusted to 50° C.

This procedure was repeated 3 times (addition of acetone and its evaporation) until the solution was stable.

The two vials were merged together.

Following the combining of these two solutions, the materials sedimented slightly. Acetone was added and evaporation of the solvent was repeated.

Before mixing the vials (3 ml+2 ml) the first solution prepared in the experiment as described in part 2, hereinabove was incubated at 9° C. over night so as to ensure the solution reached and maintained equilibrium. By doing so, the already dissolved material should not sediment. The following morning the solution's absorption was established and a different graph was obtained (FIG. 29). Following merging of the two vials, absorption measurements were performed again because the material sediment slightly. As a result of the partial sedimentation, the solution was diluted 1:1 by the addition of acetone (5 ml) and subsequently evaporation of the solution was performed at 50° C. on a hot plate. The spectrophotometer read-out of the solution, while performing the evaporation procedure changed due to the presence of acetone (FIG. 30). These experiments imply that when there is a trace of acetone it might affect the absorption readout is received.

B. Solubilizing Daunorubicine (Cerubidine Hydrochloride)

Required concentration: 2 mg/ml

Materials and Methods

2 mg Daunorubicine+1 ml Neowater™ was prepared in one vial and 2 mg of Daunorubicine+1 ml RO was prepared in a second vial.

Results

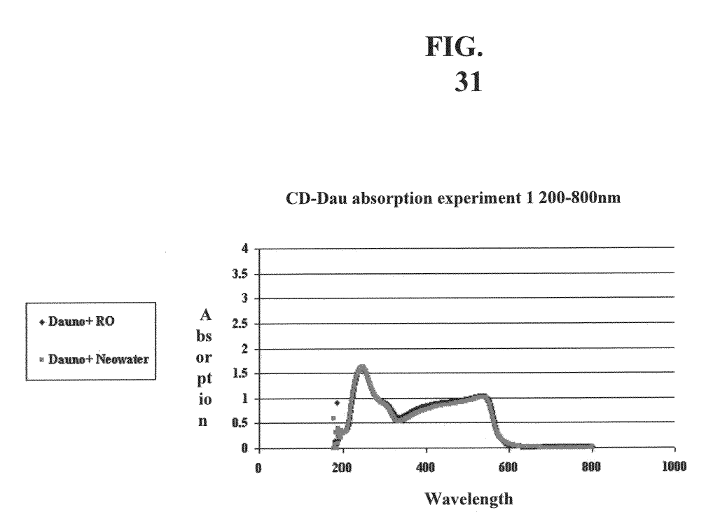

The material dissolved easily both in Neowater™ and RO as illustrated by the spectrophotometer measurements (FIG. 31).

Conclusion

Daunorubicine dissolves without difficulty in Neowater™ and RO.

C. Solubilizing t-boc

Required concentration: 4 mg/ml

Materials and Methods

1.14 ml of EtOH was added to one glass vial containing 18.5 mg of t-boc (an oily material). This was then divided into two vials and 1.74 ml Neowater™ or RO water was added to the vials such that the solution comprised 25% EtOH. Following spectrophotometer measurements, the solvent was evaporated from the solution and Neowater™ was added to both vials to a final volume of 2.31 ml in each vial. The solutions in the two vials were merged to one clean vial and packaged for shipment under vacuum conditions.

Results

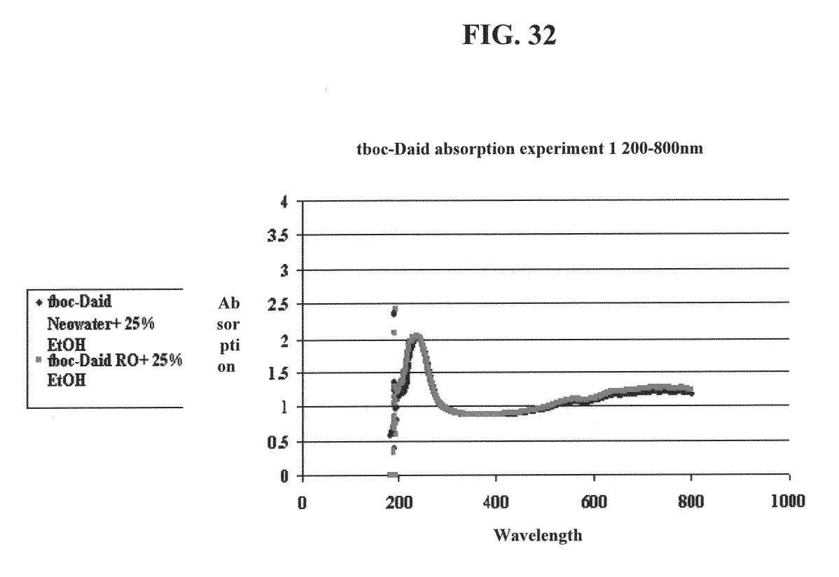

The spectrophotometer measurements are illustrated in FIG. 32. The material dissolved in ethanol. Following addition of Neowater™ and subsequent evaporation of the solvent with heat (50° C.), the material could be dissolved in Neowater™.

Conclusions

The optimal method to dissolve the materials was first to dissolve the material with a solvent (Acetone, Acetic-Acid or Ethanol) followed by the addition of the hydrophilic fluid (Neowater™) and subsequent removal of the solvent by heating the solution and evaporating the solvent.

### Example 16

### Capability of the Carrier Comprising Nanostructures to Dissolve AG-14A and AG-14B

The following experiments were performed in order to ascertain whether the carrier composition comprising nanostructures was capable of dissolving two herbal materials—AG-14A and AG-14B, both of which are known not to dissolve in water at a concentration of 25 mg/ml.

Part 1

Materials and Methods

2.5 mg of each material (AG-14A and AG-14B) was diluted in either Neowater™ alone or a solution comprising 75% Neowater™ and 25% ethanol, such that the final concentration of the powder in each of the four tubes was 2.5 mg/ml. The tubes were vortexed and heated to 50° C. so as to evaporate the ethanol.

Results

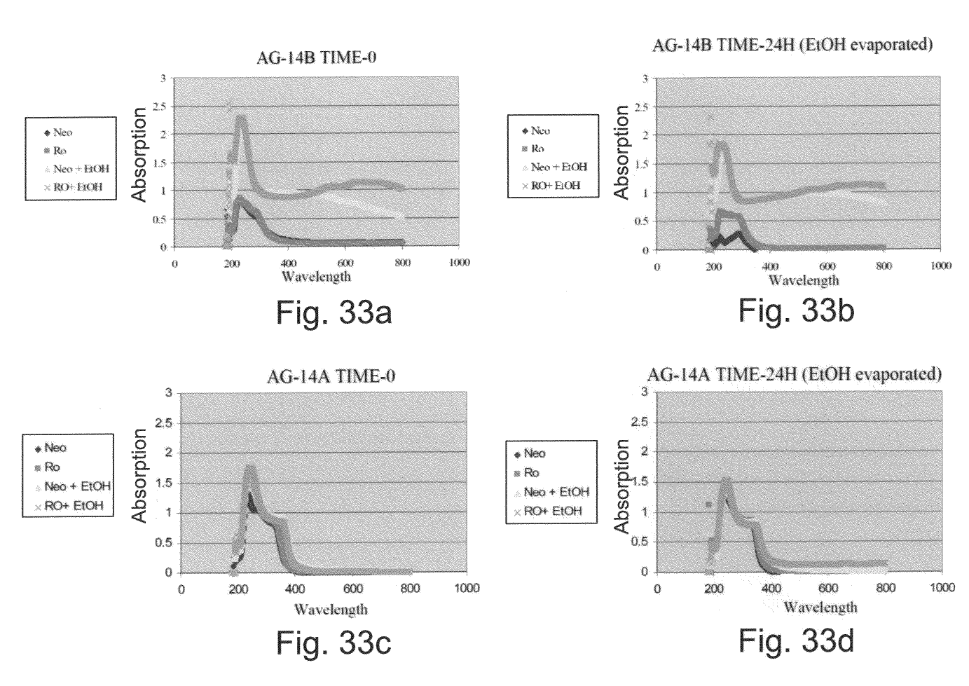

The spectrophotometric measurements of the two herbal materials in Neowater™ in the presence and absence of ethanol are illustrated in FIGS. 33A-D.

Conclusion

Suspension in RO did not dissolve of AG-14B. Suspension of AG-14B in Neowater™ did not aggregate, whereas in RO water, it did.

AG-14A and AG-14B did not dissolve in Neowater/RO.

Part 2

Material and Methods

5 mg of AG-14A and AG-14B were diluted in 62.5 μl EtOH+187.5 μl Neowater™. A further 62.5 μl of Neowater™ were added. The tubes were vortexed and heated to 50° C. so as to evaporate the ethanol.

Results

Suspension in EtOH prior to addition of Neowater™ and then evaporation thereof dissolved AG-14A and AG-14B.

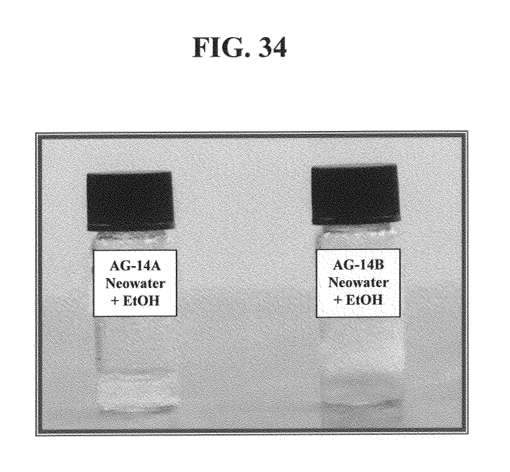

As illustrated in FIG. 34, AG-14A and AG-14B remained stable in suspension for over 48 hours.

### Example 17

### Capability of the Carrier Comprising Nanostructures to Dissolve Peptides

The following experiments were performed in order to ascertain whether the carrier composition comprising nanostructures was capable of dissolving 7 cytotoxic peptides, all of which are known not to dissolve in water. In addition, the effect of the peptides on Skov-3 cells was measured in order to ascertain whether the carrier composition comprising nanostructures influenced the cytotoxic activity of the peptides.

Materials and Methods

Solubilization: All seven peptides (Peptide X, X-5FU, NLS-E, Palm-PFPSYK (CMFU), PFPSYKLRPG-NH₂, NLS-p2-LHRH, and F-LH-RH-palm kGFPSK) were dissolved in Neowater™ at 0.5 mM. Spectrophotometric measurements were taken.

In Vitro Experiment: Skov-3 cells were grown in McCoy's 5A medium, and diluted to a concentration of 1500 cells per well, in a 96 well plate. After 24 hours, 2 μl (0.5 mM, 0.05 mM and 0.005 mM) of the peptide solutions were diluted in 1 ml of McCoy's 5A medium, for final concentrations of 10⁻⁶ M, 10⁻⁷ M and 10⁻⁸ M respectively. 9 repeats were made for each treatment. Each plate contained two peptides in three concentration, and 6 wells of control treatment. 90 μl of McCoy's 5A medium+peptides were added to the cells. After 1 hour, 10 μl of FBS were added (in order to prevent competition). Cells were quantified after 24 and 48 hours in a viability assay based on crystal violet. The dye in this assay, stains DNA. Upon solubilization, the amount of dye taken up by the monolayer was quantified in a plate reader.

Results

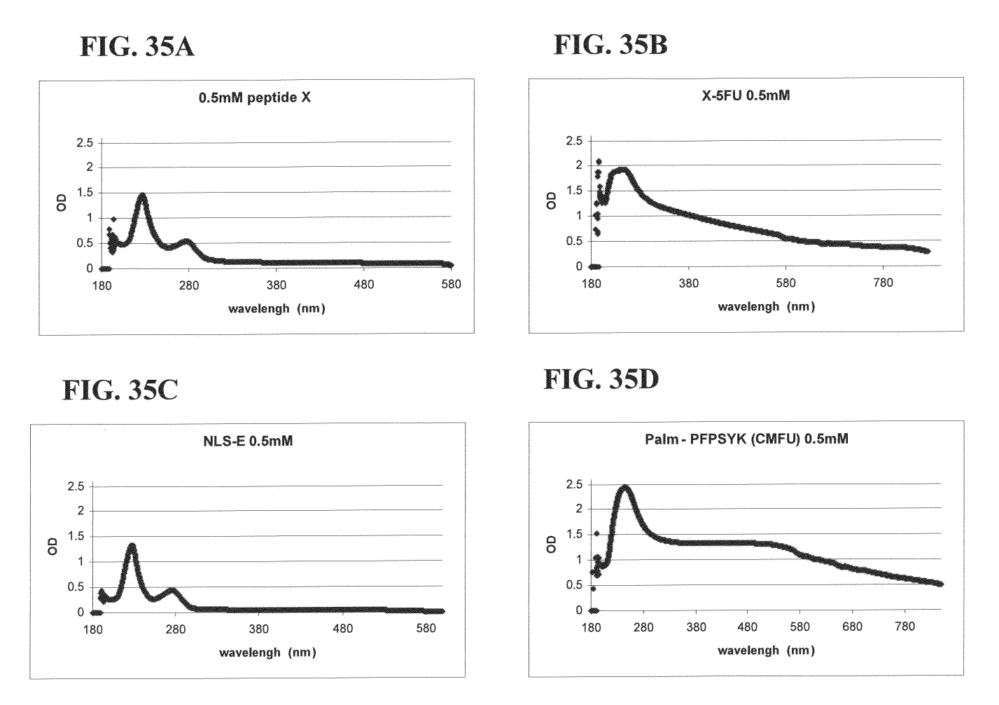

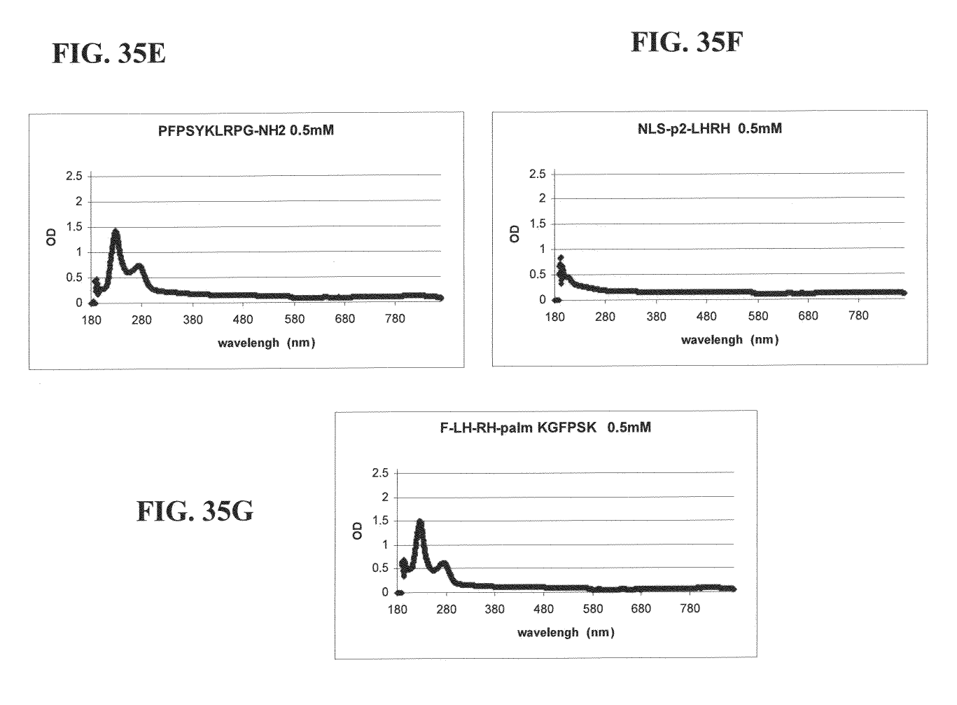

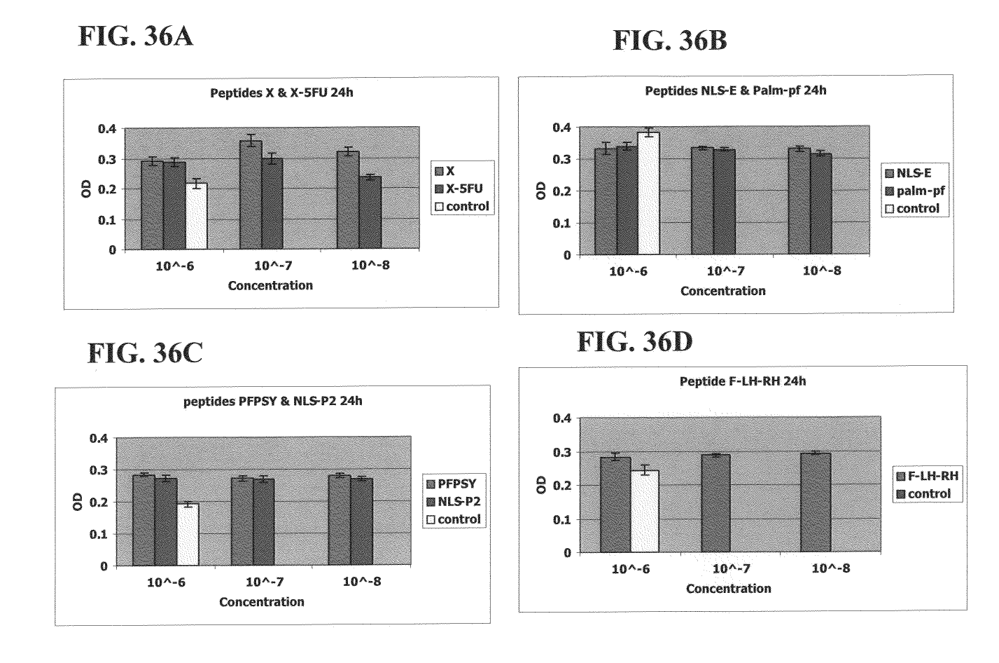

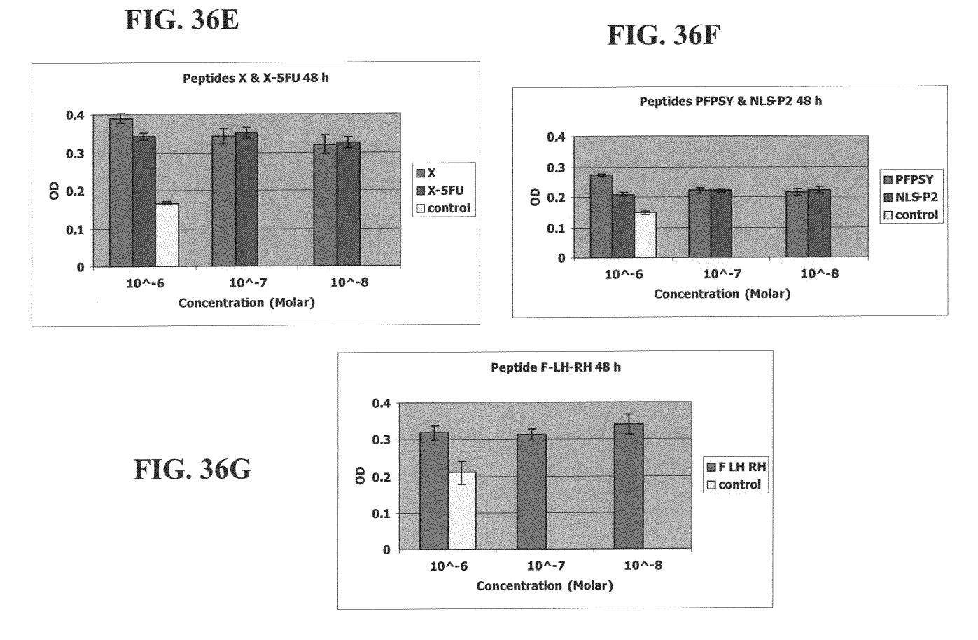

The spectrophotometric measurements of the 7 peptides diluted in Neowater™ are illustrated in FIGS. 35A-G. As illustrated in FIGS. 36A-G, all the dissolved peptides comprised cytotoxic activity.

### Example 18

### Capability of the Carrier Comprising Nanostructures to Dissolve Retinol

The following experiments were performed in order to ascertain whether the carrier composition comprising nanostructures was capable of dissolving retinol.

Materials and Methods

Retinol (vitamin A) was purchased from Sigma (Fluka, 99% HPLC). Retinol was solubilized in Neowater™ under the following conditions.

1% retinol (0.01 gr in 1 ml) in EtOH and Neowater™

0.5% retinol (0.005 gr in 1 ml) in EtOH and Neowater™

0.5% retinol (0.125 gr in 25 ml) in EtOH and Neowater™.

0.25% retinol (0.0625 gr in 25 ml) in EtOH and Neowater™. Final EtOH concentration: 1.5%

Absorbance spectrum of retinol in EtOH: Retinol solutions were made in absolute EtOH, with different retinol concentrations, in order to create a calibration graph; absorbance spectrum was detected in a spectrophotometer.

2 solutions with 0.25% and 0.5% retinol in Neowater™ with unknown concentration of EtOH were detected in a spectrophotometer. Actual concentration of retinol is also unknown since some oil drops are not dissolved in the water.

Filtration: 2 solutions of 0.25% retinol in Neowater™ were prepared, with a final EtOH concentration of 1.5%. The solutions were filtrated in 0.44 and 0.2 μl filter.

Results

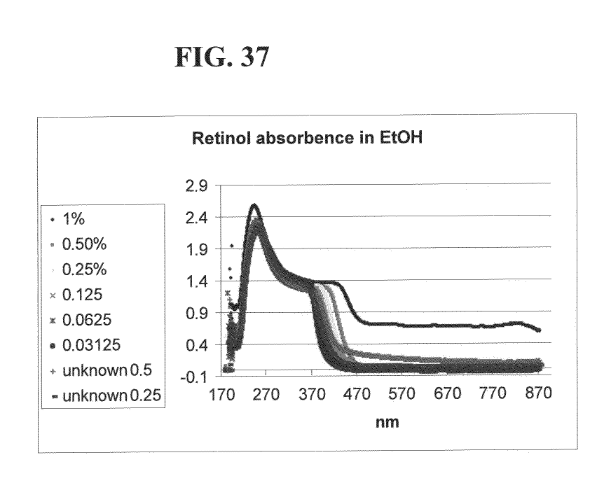

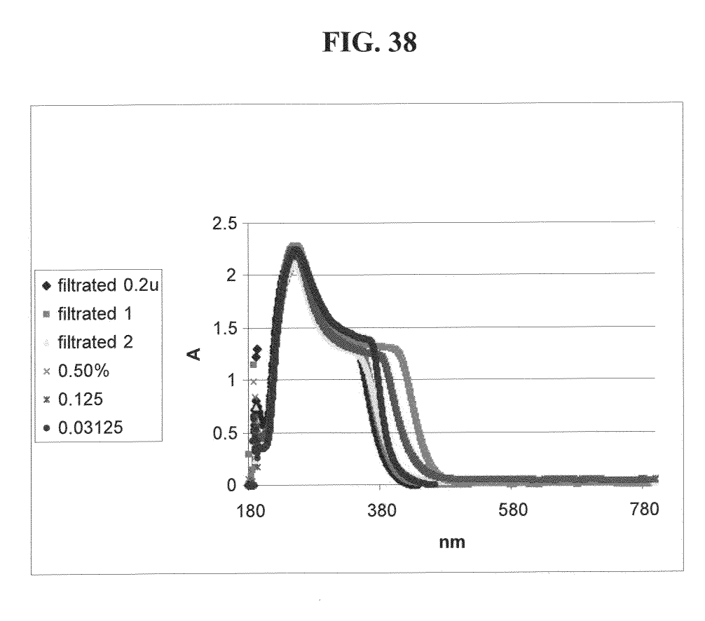

Retinol solubilized easily in alkali Neowater™ rather than acidic Neowater™. The color of the solution was yellow, which faded over time. In the absorbance experiments, 0.5% retinol showed a similar pattern to 0.125% retinol, and 0.25% retinol shows a similar pattern to 0.03125% retinol—see FIG. 37. Since Retinol is unstable in heat; (its melting point is 63° C.), it cannot be autoclaved. Filtration was possible when retinol was fully dissolved (in EtOH). As illustrated in FIG. 38, there is less than 0.03125% retinol in the solutions following filtration. Both filters gave similar results.

### Example 19

### Capability of the Carrier Comprising Nanostructures to Dissolve Material X

The following experiments were performed in order to ascertain whether the carrier composition comprising nanostructures was capable of dissolving material X at a final concentration of 40 mg/ml.

Part 1—Solubility in Water and DMSO

Materials and Methods

In a first test tube, 25 μl of Neowater™ was added to 1 mg of material “X”. In a second test tube 25 μl of DMSO was added to 1 mg of material “X”. Both test tubes were vortexed and heated to 60° C. and shaken for 1 hour on a shaker.

Results

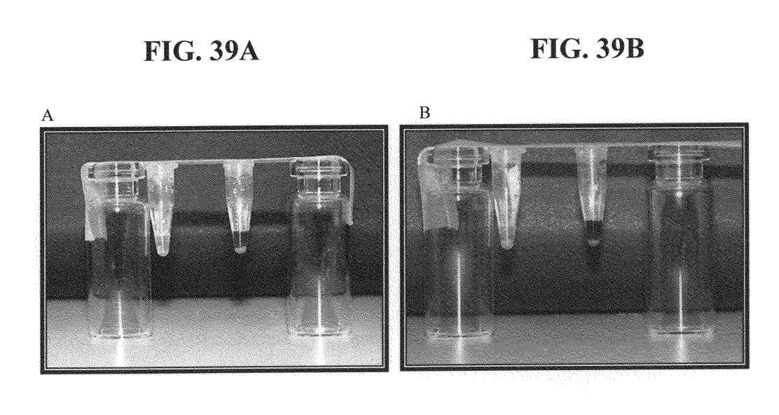

The material did not dissolve at all in Neowater™ (test tube 1). The material dissolved in DMSO and gave a brown-yellow color. The solutions remained for 24-48 hours and their stability was analyzed over time (FIG. 39A-B).

Conclusions

Neowater™ did not dissolve material “X” and the material sedimented, whereas DMSO almost completely dissolved material “X”.

Part 2—Reduction of DMSO and Examination of the Material Stability/Kinetics in Different Solvents Over the Course of Time.

Materials and Methods

6 different test tubes were analyzed each containing a total reaction volume of 25 μl:

1. 1 mg “X”+25 μl Neowater™ (100%).

2. 1 mg “X”+12.5 μl DMSO [symbol] 12.5 μl Neowater™ (50%).

3. 1 mg “X”+12.5 μl DMSO+12.5 μl Neowater™ (50%).

4. 1 mg “X”+6.25 μl DMSO+18.75 μl Neowater™ (25%).

5. 1 mg “X”+25 μl Neowater™+sucrose*(10%).

6. 1 mg+12.5 μl DMSO+12.5 μl dehydrated Neowater™**(50%).

*0.1 g sucrose+ml (Neowater™)=10% Neowater™+sucrose
**Dehydrated Neowater™ was achieved by dehydration of Neowater™ for 90 min at 60° C.

All test tubes were vortexed, heated to 60° C. and shaken for 1 hour.

Results

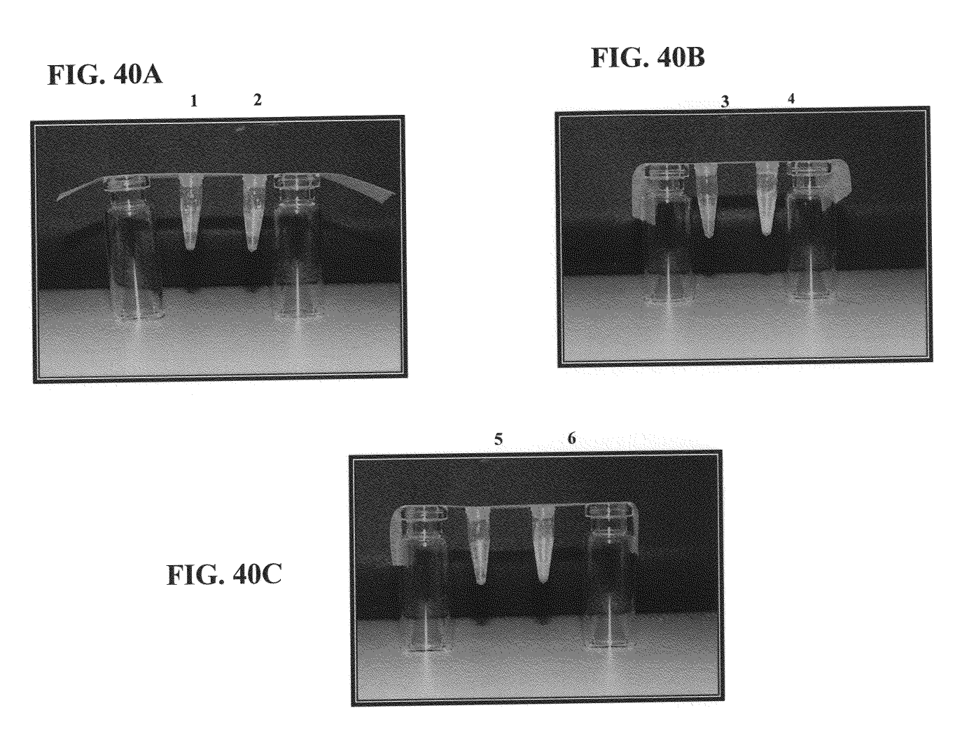

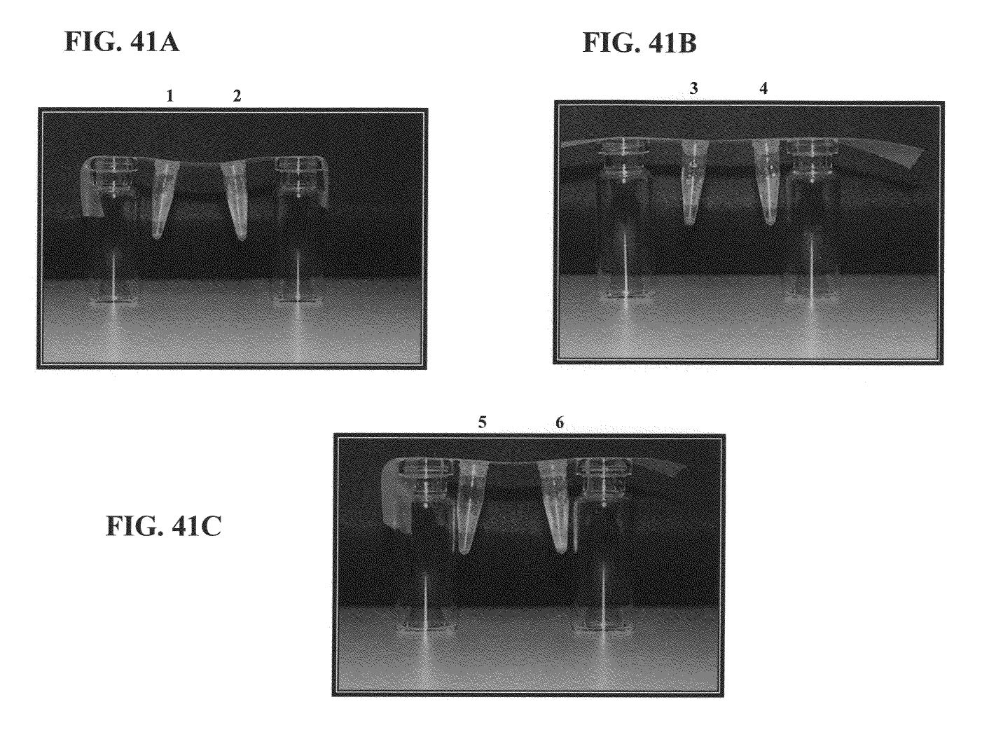

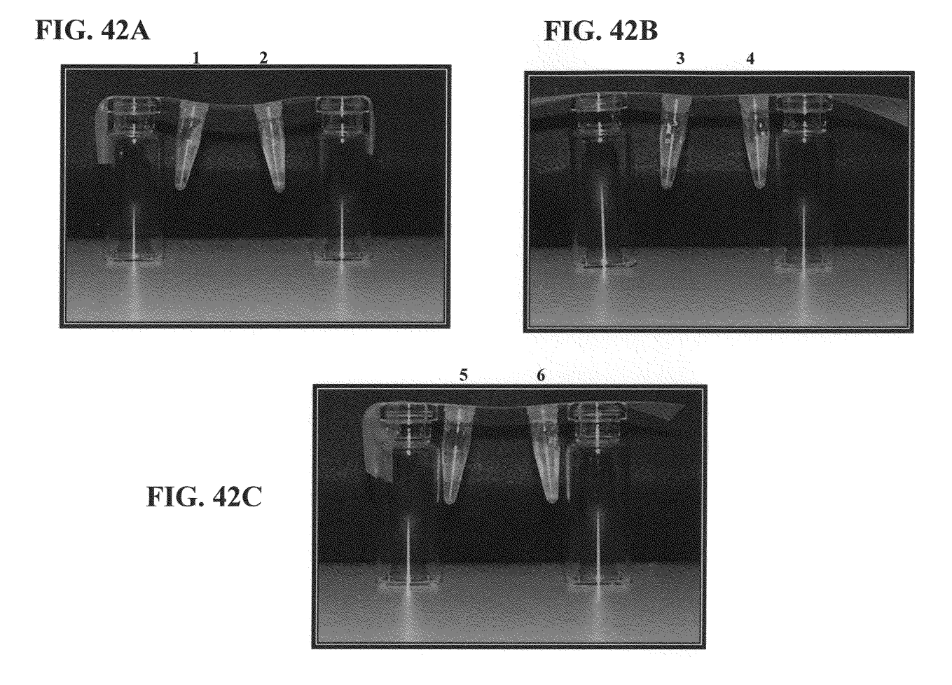

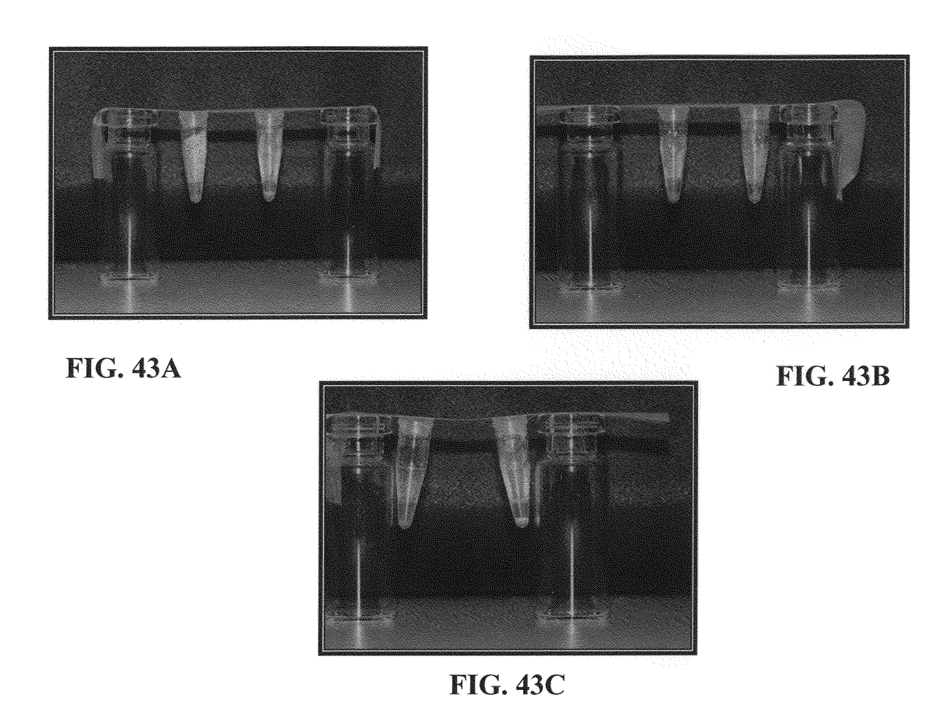

The test tubes comprising the 6 solvents and substance X at time 0 are illustrated in FIGS. 40A-C. The test tubes comprising the 6 solvents and substance X at 60 minutes following solubilization are illustrated in FIGS. 41A-C. The test tubes comprising the 6 solvents and substance X at 120 minutes following solubilization are illustrated in FIGS. 42A-C. The test tubes comprising the 6 solvents and substance X 24 hours following solubilization are illustrated in FIGS. 43A-C.

Conclusion

Material “X” did not remain stable throughout the course of time, since in all the test tubes the material sedimented after 24 hours.

There is a different between the solvent of test tube 2 and test tube 6 even though it contains the same percent of solvents. This is because test tube 6 contains dehydrated Neowater™ which is more hydrophobic than non-dehydrated Neowater™.

Part 3 Further Reduction of DMSO and Examination of the Material Stability/Kinetics in Different Solvents Over the Course of Time.

Materials and Methods

1 mg of material “X”+50 μl DMSO were placed in a glass tube. 50 μl of Neowater™ were titred (every few seconds 5 μl) into the tube, and then 500 μl of a solution of Neowater™ (9% DMSO+91% Neowater™) was added.

In a second glass tube, 1 mg of material “X”+50 μl DMSO were added. 50 μl of RO were titred (every few seconds 5 μl) into the tube, and then 500 μl of a solution of RO (9% DMSO+91% RO) was added.

Results

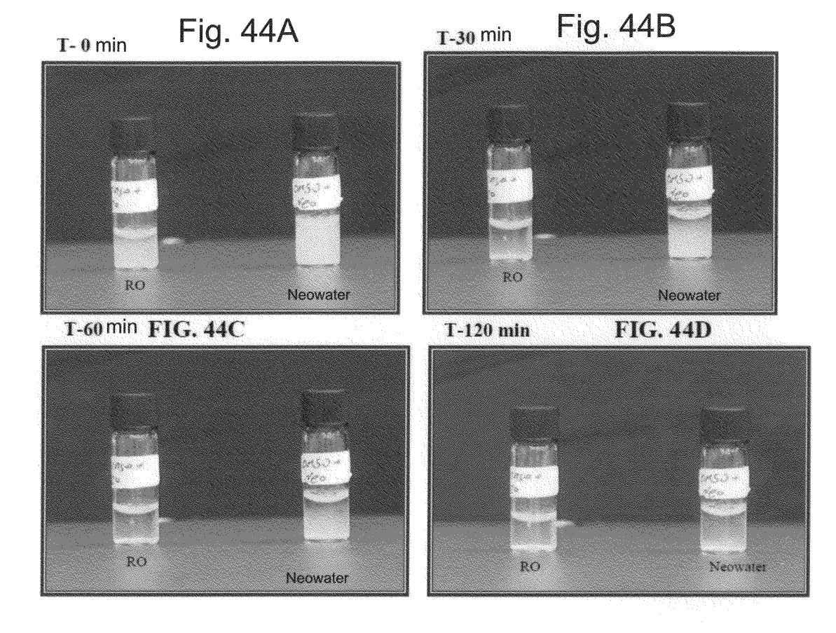

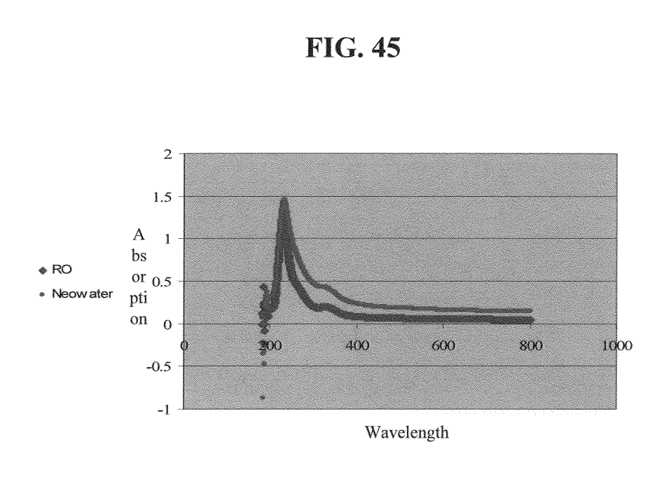

As illustrated in FIGS. 44A-D, material “X” remained dispersed in the solution comprising Neowater™, but sedimented to the bottom of the tube, in the solution comprising RO water. FIG. 45 illustrates the absorption characteristics of the material dispersed in RO/Neowater™ and acetone 6 hours following vortexing.

Conclusion

It is clear that material “X” dissolves differently in RO compare to Neowater, and it is more stable in Neowater™ compare to RO. From the spectrophotometer measurements (FIG. 45), it is apparent that the material “X” dissolved better in Neowater™ even after 5 hours, since, the area under the graph is larger than in RO. It is clear the Neowater™ hydrates material “X”. The amount of DMSO may be decreased by 20-80% and a solution based on Neowater™ may be achieved that hydrates material “X” and disperses it in the Neowater™.

### Example 20

### Capability of the Carrier Comprising Nanostructures to Dissolve SPL 2101 and SPL 5217

The following experiments were performed in order to ascertain whether the carrier composition comprising nanostructures was capable of dissolving material SPL 2101 and SPL 5217 at a final concentration of 30 mg/ml.

Materials and Methods

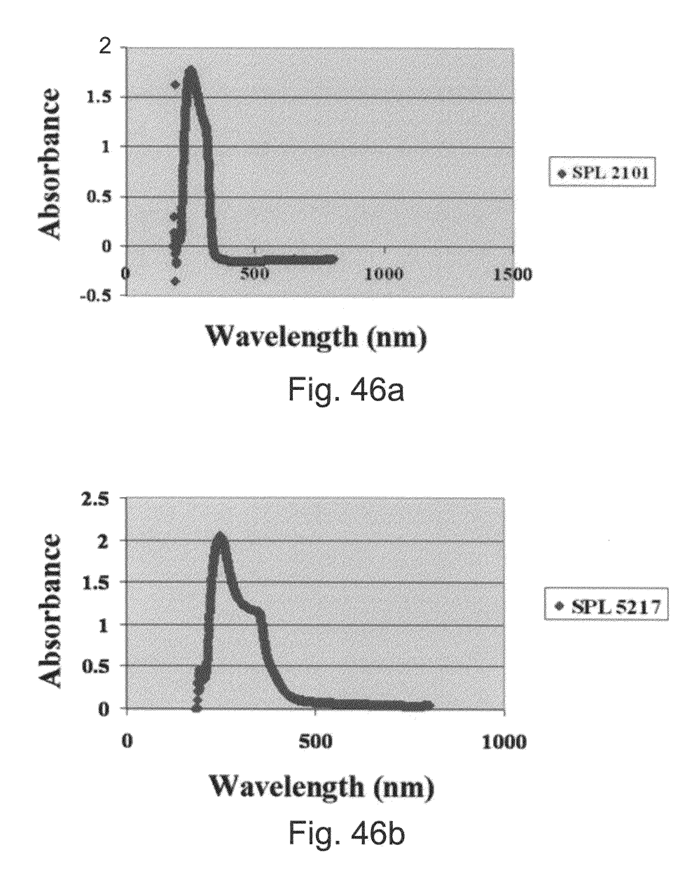

SPL 2101 was dissolved in its optimal solvent (ethanol)-FIG. 46A and SPL 5217 was dissolved in its optimal solvent (acetone)-FIG. 46B. The two compounds were put in glass vials and kept in dark and cool environment. Evaporation of the solvent was performed in a dessicator and over a long period of time Neowater™ was added to the solution until there was no trace of the solvents.

Results

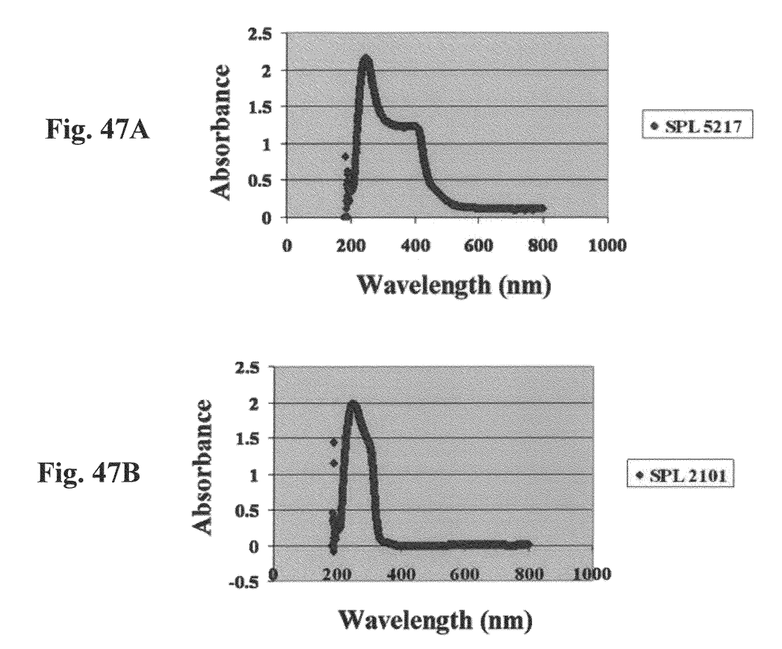

SPL 2101 & SPL 5217 dissolved in Neowater™ as illustrated by the spectrophotometer data in FIGS. 47A-B.

### Example 21

### Capability of the Carrier Comprising Nanostructures to Dissolve Taxol

The following experiments were performed in order to ascertain whether the carrier composition comprising nanostructures was capable of dissolving material taxol (Paclitaxel) at a final concentration of 0.5 mM.

Materials and Methods

Solubilization: 0.5 mM Taxol solution was prepared (0.0017 gr in 4 ml) in either DMSO or Neowater™ with 17% EtOH. Absorbance was detected with a spectrophotometer.

Cell viability assay: 150,000 293T cells were seeded in a 6 well plate with 3 ml of DMEM medium. Each treatment was grown in DMEM medium based on RO or Neowater™. Taxol (dissolved in Neowater™ or DMSO) was added to final concentration of 1.666 μM (10 μl of 0.5 mM Taxol in 3 ml medium). The cells were harvested following a 24 hour treatment with taxol and counted using trypan blue solution to detect dead cells.

Results

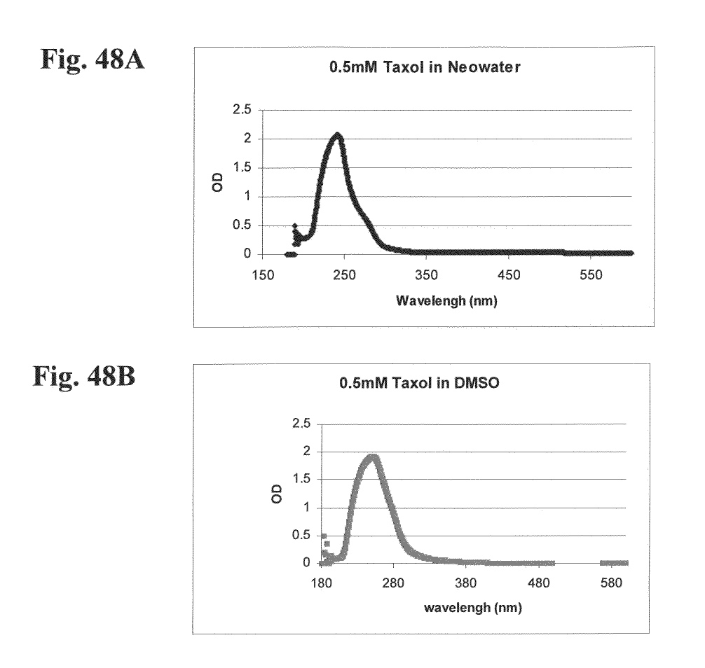

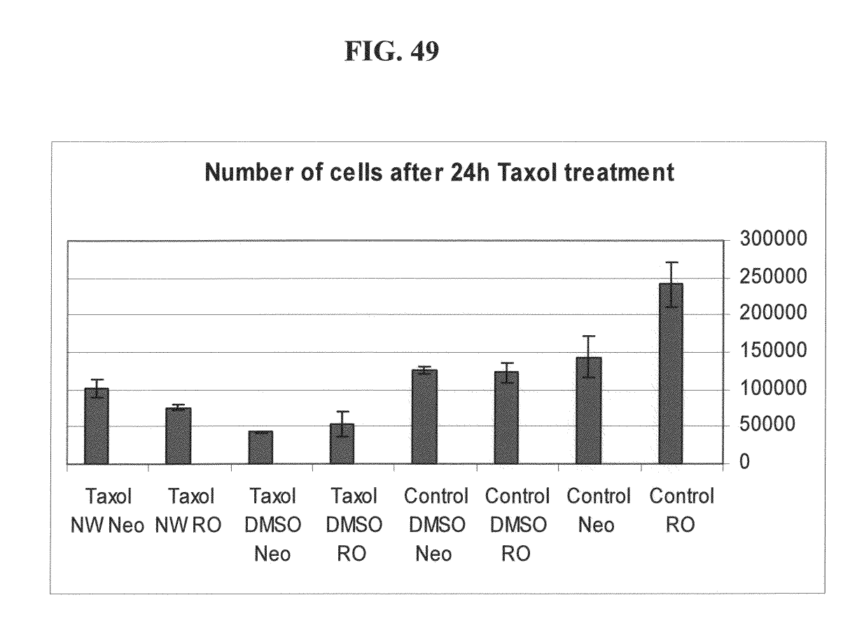

Taxol dissolved both in DMSO and Neowater™ as illustrated in FIGS. 48A-B. The viability of the 293T cells following various solutions of taxol is illustrated in FIG. 49.

Conclusion

Taxol comprised a cytotoxic effect following solution in Neowater™.

### Example 22

### Stabilizing Effect of the Carrier Comprising Nanostructures

The following experiment was performed to ascertain if the carrier composition comprising nanostructures effected the stability of a protein.

Materials and Methods

Two commercial Taq polymerase enzymes (Peq-lab and Bio-lab) were checked in a PCR reaction to determine their activities in ddH₂O(RO) and carrier comprising nanostructures (Neowater™—Do-Coop technologies, Israel). The enzyme was heated to 95° C. for different periods of time, from one hour to 2.5 hours.

2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

As illustrated in FIGS. 50A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 23

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

5 Three samples were set up and placed in a PCR machine at a constant temperature of 95° C. Incubation time was: 60, 75 and 90 minutes.

Following boiling of the Taq enzyme the following components were added:

2.5 μl 10× reaction buffer Y (Peq-lab)
0.5 μl dNTPs 10 mM (Bio-lab)
1 μl primer GAPDH mix 10 pmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/μl)

Five samples were set up and placed in a PCR machine at a constant temperature of 95° C. Incubation time was: 60, 75, 90 120 and 150 minutes.

Following boiling of the Taq enzyme the following components were added:

2.5 μl TAQ 10× buffer Mg-free (Bio-lab)

### 1.5 μl MgCl₂ 25 mM (Bio-lab)

0.5 μl dNTPs 10 mM (Bio-lab)
1 μl primer GAPDH mix (10 pmol/μl)
0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR Program:

1. 94° C. 2 minutes denaturation

2. 94° C. 30 seconds denaturation

3.60° C. 30 seconds annealing

4. 72° C. 30 seconds elongation

repeat steps 2-4 for 30 times

5. 72° C. 10 minutes elongation

Results

As illustrated in FIG. 51, the carrier composition comprising nanostructures protected both the enzymes from heat stress for up to 1.5 hours.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A pharmaceutical composition comprising:
(a) at least one pharmaceutical agent as an active ingredient;
(b) nanostructures and liquid, wherein said nanostructures comprise a core material of a nanometric size enveloped by ordered fluid molecules of said liquid, said core material and said envelope of ordered fluid molecules being in a steady physical state and whereas said nanostructures and liquid being formulated to enhance in vivo uptake of said at least one pharmaceutical agent.

2. A method of enhancing in vivo uptake of a pharmaceutical agent into a cell comprising administering to an individual the pharmaceutical agent in combination with nanostructures and liquid, wherein said nanostructures comprise a core material of a nanometric size enveloped by ordered fluid molecules of said liquid, said core material and said envelope of ordered fluid molecules being in a steady physical state and whereas said nanostructures and liquid being formulated to enhance in vivo uptake the pharmaceutical agent, thereby enhancing in vivo uptake of the pharmaceutical agent into the cell.

3. The pharmaceutical composition of claim 1, wherein said pharmaceutical agent is a therapeutic agent, cosmetic agent or a diagnostic agent.

4-5. (canceled)

6. The pharmaceutical composition of claim 1, wherein said pharmaceutical agent is selected from the group consisting of a protein agent, a nucleic acid agent, a small molecule agent, a cellular agent and a combination thereof.

7-27. (canceled)

28. The pharmaceutical composition of claim 1, wherein said nanostructures are formulated from hydroxyapatite.

29. The pharmaceutical composition of claim 1, wherein said pharmaceutical agent is selected to treat a skin condition.

30. (canceled)

31. The pharmaceutical composition of claim 1, formulated in a topical composition.

32. The pharmaceutical composition of claim 1, wherein said pharmaceutical agent is selected to treat or diagnose a brain condition.

33. (canceled)

34. The method of claim 2, wherein the cell is a mammalian cell, a bacterial cell or a viral cell.

35. The method of claim 2, wherein said pharmaceutical agent is a therapeutic agent, cosmetic agent or a diagnostic agent.

36. The method of claim 2, wherein said pharmaceutical agent is selected from the group consisting of a protein agent, a nucleic acid agent, a small molecule agent, a cellular agent and a combination thereof.

37. The method of claim 2, wherein said nanostructures are formulated from hydroxyapatite.

38. The method of claim 2, wherein said pharmaceutical agent is selected to treat a skin condition.

39. The method of claim 2, wherein said pharmaceutical agent is selected to treat or diagnose a brain condition.

40. A pharmaceutical composition comprising:
(a) taxol as an active ingredient; and
(b) nanostructures and liquid, wherein said nanostructures comprise a core material of a nanometric size enveloped by ordered fluid molecules of said liquid, said core material and said envelope of ordered fluid molecules being in a steady physical state.

41. The pharmaceutical composition of claim 40, being formulated for oral delivery.
